(19) United States
(12) Patent Application Publication
(10) Pub. No.: US 2024/0317795 A1
(43) Pub. Date: Sep. 26, 2024

(54) HALOGENATED CHOLESTEROL ANALOGUES AND METHODS OF MAKING AND USING SAME

(71) Applicants: THE REGENTS OF THE UNIVERSITY OF MICHIGAN, Ann Arbor, MI (US); THE UNITED STATES GOVERNMENT AS REPRESENTED BY THE DEPARTMENT OF VETERANS AFFAIRS, Washington, DC (US)

(72) Inventors: Benjamin L. Viglianti, Ann Arbor, MI (US); Allen F. Brooks, Ann Arbor, MI (US); Peter J. H. Scott, Ypsilanti, MI (US); Stephen Thompson, Ann Arbor, MI (US); Stefan Verhoog, Ann Arbor, MI (US); Milton D. Gross, Ann Arbor, MI (US); Wade P. Winton, Ann Arbor, MI (US)

(21) Appl. No.: 18/677,767

(22) Filed: May 29, 2024

Related U.S. Application Data
(63) Continuation of application No. 17/250,891, filed on Mar. 19, 2021.
(60) Provisional application No. 62/841,793, filed on May 1, 2019.
(60) Provisional application No. 62/738,090, filed on Sep. 28, 2018.

(51) Int. Cl. C07J 9/00; C07J 21/00

(52) U.S. Cl. CPC C07J 9/00; C07J 21/00; C07B 2200/05

(57) ABSTRACT

Provided herein are halogenated cholesterol analogues, including methods of making and using the same. Also provided are methods of making radiolabeled cholesterol analogues including admixing an epoxide with a fluorine-18 source under conditions to form a radiofluorinated cholesterol analogue.

### STATEMENT OF GOVERNMENT SUPPORT

This invention was made with government support under EB021155 awarded by National Institutes of Health. The government has certain rights in the invention.

### BACKGROUND

Medical imaging techniques, such as Single Photon Emission Computed Tomography (SPECT) and Positron Emission Tomography (PET), are useful tools in internal diagnostic medicine. These techniques utilize radionuclide containing contrast agents, detected by complex detectors that are combined with computational techniques to develop three-dimensional images of internal organs and features. Generally speaking, PET provides imaging that is significantly higher resolution than SPECT (5-7 mm compared to 12-15 mm, respectively). Additionally, PET has recently been adapted to enable quantification of medical imaging, which has not been accomplished with SPECT.

Iodine-131 is a relatively common radionuclide that is used for SPECT based imaging. Iodine-131, having a half-life of about 8 days, is often used for therapeutic applications, such as to treat hyperthyroidism or thyroid cancers. Iodine-124 is also useful as a PET probe.

The most commonly used radioisotope for PET is fluorine-18, which offers the advantages of high resolution imaging (about 2.5 mm in tissue) and minimal perturbation of radioligand binding. Despite these advantages, the development of novel ¹⁸F radiotracers is currently impeded by a paucity of general and effective radiofluorination methods, particularly in view of the relatively short half-life of ¹⁸F (t_{1/2}=110 minutes). There are currently few robust synthetic procedures for the incorporation of ¹⁸F into organic molecules with sufficient speed, selectivity, yield, radiochemical purity, and reproducibility to provide clinical imaging materials. Direct methods for the late stage nucleophilic [¹⁸F]fluorination of electron-rich aromatic substrates remains an especially long-standing challenge in the PET community.

I-131-6B-iodomethyl-19-norcholest-5-(10)-en-3B-ol (“NP-59”), the structure of which is shown below, is a cholesterol analogue developed in the 1970s that has traditionally been used for SPECT-imaging applications. As it is a cholesterol analogue, NP-59 can accumulate in tissues and features that are rich in cholesterol.

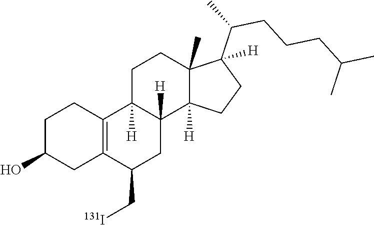

One use for NP-59 is medical imaging of the adrenal cortex, particularly in the case of identifying adrenal adenomas. The adrenal cortex mediates the stress response by producing the stress response hormones glucocorticoid and mineralocorticoid from the precursor cholesterol. Thus, the cortex requires significant uptake of cholesterol, which enables the use of radiotracer labeled cholesterol analogues, such as NP-59, in imaging of the cortex.

Adrenal adenomas are benign tumors on the adrenal cortex that are frequently yellow and waxy in color, as a result of the excessive uptake and storage of cholesterol within the tumor. These tumors overproduce the steroids glucocorticoid and mineralocorticoid, which may result in Cushing's syndrome in some cases. Imaging of the adenomas is enabled by excessive uptake and storage of cholesterol analogues such as NP-59.

Vulnerable plaques are a collection of white blood cells and lipids, including cholesterol, that accumulate on the walls of arteries. The plaques are generally unstable and prone to rupturing, which can have dire health consequences such as heart attack or stroke.

Effective identification and monitoring of these plaques could provide for significantly enhanced health outcomes as this may allow for earlier intervention in the case of troublesome plaques.

Detection of these plaques has been historically difficult as common cardiac techniques like stress tests or angiography tend not to be capable of identifying them.

Intravascular ultrasound, thermography, near-infrared spectroscopy, and cardiac CT angiography have become increasingly common in identifying these plaques.

Given the prevalence of cholesterol within these plaques, cholesterol-analogue radiotracer biomolecules may provide an attractive avenue for imaging plaques with advanced techniques, such as SPECT or PET.

### SUMMARY

In a first aspect, the present disclosure provides a compound having the structure of Formula (I):

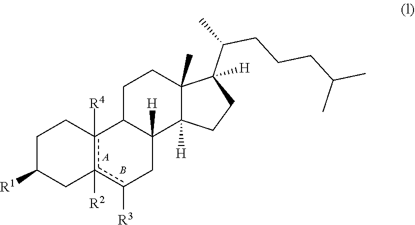

wherein:
R¹ is OH or OP;
R², when present, is OH or X;
R³ is H, OH, X, CH₂—X, or CH₂-LG;
R⁴, when present, is C₁₋₆ alkyl, C₁₋₆ alkylene-X, or C₁₋₆ alkylene-LG;
X is a halogen;
P is an alcohol protecting group; and
LG is a leaving group;
each of bond A and bond B is a single or a double bond and only one of bond A and bond B can be a double bond;
   with the proviso that:
at least one X or LG is present; and if LG is present, R¹ is OP;
if one of R² and R³ is F and the other OH, then the F is ¹⁸F; and the compound is not:

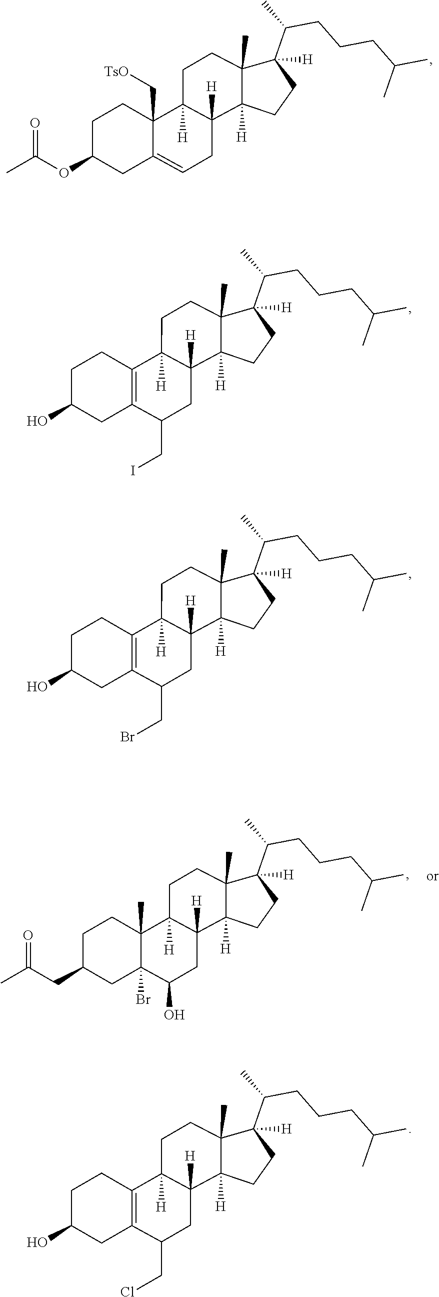

In another aspect, the disclosure provides a method of preparing a compound having the structure of Formula (II)

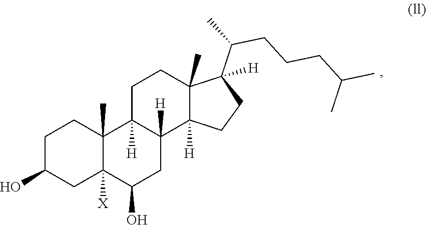

wherein X is ¹⁸F, ⁷⁶Br, or ⁷⁷Br, comprising admixing 5,6-epoxycholesterol and a radiolabeled source under conditions sufficient to form the compound of Formula (II).

In yet another aspect, the disclosure provides a method comprising admixing an epoxide with a metal catalyst and a fluorine-18 source to form a α,β-hydroxy fluoride compound, wherein the fluorine-18 source comprises H-¹⁸F.

In another aspect, the disclosure provides a method comprising admixing cholesterol and pivaloyl chloride to form cholest-5-en-3-pivaloate; reacting cholest-5-en-3-pivaloate with N-bromoacetamide to form a 5-bromocholestan-6-hydroxy-3-pivaloate; reacting the 5-bromocholestan-6-hydroxy-3-pivaloate with lead tetraacetate to form a 5-bromocholestan-6(19)-oxo-3-pivaloate; reacting 5-bromocholestan-6(19)-oxo-3-pivaloate with activated zinc to form a cholest-5-en-19-hydroxy-3-pivaloate; reacting the cholest-5-en-19-hydroxy-3-pivaloate with mesyl chloride then potassium acetate to form (3S,5R,10S,13R,17R)-6-hydroxy-13-methyl-17-((R)-6-methylheptan-2-yl)tetradecahydro-6H-5,10-methanocyclopenta[a]phenanthren-3-yl pivaloate; and reacting (3S,5R,10S,13R,17R)-6-hydroxy-13-methyl-17-((R)-6-methylheptan-2-yl)tetradecahydro-6H-5,10-methanocyclopenta[a]phenanthren-3-yl pivaloate with boron trifluoride and methanesulfonic acid to form 6-methyl(methanesulfonyl)-19-norcholest-5(10)-en-3-yl pivaloate.

Further aspects and advantages will be apparent to those of ordinary skill in the art from a review of the following detailed description. The description hereafter includes specific embodiments with the understanding that the disclosure is illustrative, and is not intended to limit the invention to the specific embodiments described herein.

### BRIEF DESCRIPTION OF THE DRAWINGS

FIG. 1 shows PET images taken 60 minutes after injection of a BL6 control mouse and an ApoE mouse with ¹⁸F-radiolabeled NP-59.

### DETAILED DESCRIPTION

Provided herein are halogenated cholesterol analogues, including methods of making and using the same. In particular, the halogenated cholesterol analogues are fluorinated and iodinated, e.g., radiofluorinated and radioiodinated, cholesterol analogues.

The well-known imaging agent NP-59, an iodinated cholesterol analogue, was developed for functionally depicting the adrenal cortex and is used in the functional characterization of adenomas and carcinomas of the adrenal gland in patients with Cushing's syndrome, primary aldosteronism, hyperandrogenism, and to characterize the endocrine secretory status of otherwise “euadrenal” neoplasms. When labeled with radioiodine-131, NP-59 has an undesirably long biological half-life, with limited imaging resolution. Despite these limitations NP-59 has been in continued use in Europe and Asia.

Substitution of other iodine isotopes with single photon emission tomography (SPECT) has been used to mitigate radiation dose, but imaging protocols still require multi-day imaging protocols. PET imaging with radioiodine-124 has the benefit of PET coincidence detection with substantially improved imaging resolution, but has been limited by the low positron output of iodine-124 (¹²⁴I decays by β⁺ 26% vs ¹⁸F, 97%) leading to noise that lowers image quality, and undesirably high dosimetry. Alternatively, fluorine-18 has more favorable physical characteristics with a high percentage of decay by β⁺ while maintaining high PET imaging spatial resolution. Further, a fluorine for iodine substitution has been shown in other agent to shorter biological half-life with more rapid clearance from non-target background tissues facilitating early diagnostic quality image reconstruction and clinical image interpretation.

The compounds described herein have a structure of Formula (I):

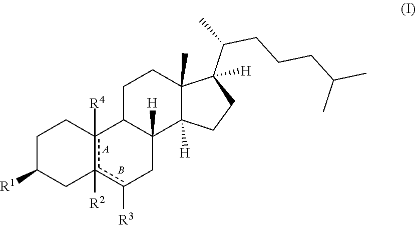

wherein the substituents are described in detail below.

The compounds described herein can be used to image cholesterol metabolism related to various pathologies. When the compounds are radio-labeled with, for example, ¹⁸F or ¹²⁴I, they can be useful for improving diagnostic accuracy, e.g., via PET imaging, image quality and shortening the procedure to one patient visit.

### Chemical Definitions

As used herein, the term “alkyl” refers to straight chained and branched saturated hydrocarbon groups. The term Cn means the alkyl group has “n” carbon atoms. For example, C4 alkyl refers to an alkyl group that has 4 carbon atoms. C1-6alkyl refers to an alkyl group having a number of carbon atoms encompassing the entire range (i.e., 1 to 6 carbon atoms), as well as all subgroups (e.g., 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, the term “alkylene” refers to a bivalent saturated aliphatic radical. The term Cn means the alkylene group has “n” carbon atoms. For example, C1-6alkylene refers to an alkylene group having a number of carbon atoms encompassing the entire range, as well as all subgroups, as previously described for “alkyl” groups.

As used herein, the term “epoxy” or “epoxide” refers to a three-membered ring whose backbone comprises two carbon atoms and an oxygen atom.

As used herein, the term “halogen” refers to fluorine, chlorine, bromine, and iodine.

In some cases, the halo is a radioactive halogen. Examples of radioactive halogens include, but are not limited to, fluorine-18, chlorine-37, bromine-77, and iodine-124, iodine-131.

As used herein, the term “leaving group” refers to any atom or moiety that is capable of being displaced by another atom or moiety in a chemical reaction. Examples of suitable leaving groups include, but are not limited to, a dialkyl ether, triflate, tosyl, mesyl, and a halogen.

As used herein, the term “alcohol protecting group” refers to a group introduced into a molecule by chemical modification of an alcohol (i.e. hydroxyl) group in order to obtain chemoselectivity in a subsequent chemical reaction and to prevent modification of the alcohol group under certain conditions. Examples of suitable alcohol protecting groups include, but are not limited to, methyl, t-butyloxycarbonyl (Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS or TBS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, .alpha.-naphthoate, nitrate, alkyl N,N,N,N′-tetramethylphosphorodiamidate, alkyl N-phenylcarbamate, borate, dimethylphosphinothioyl, alkyl 2,4-dinitrophenylsulfenate, sulfate, methanesulfonate (mesyl), benzylsulfonate, and tosyl (Ts). In some cases, the alcohol protecting group si methoxymethyl ether (MOM), tetrahydropyranyl ether (THP), t-butyl ether, allyl ether, benzyl ether, t-butyldimethylsilyl ether (TBDMS), t-butyldiphenylsilyl ether (TBDPS), acetoxy, pivalic acid ester, or benzoic acid ester. In some cases, the alcohol protecting group is MOM or THP.

### Cholesterol Analogues

Provided herein are compounds having a structure of Formula (I), wherein

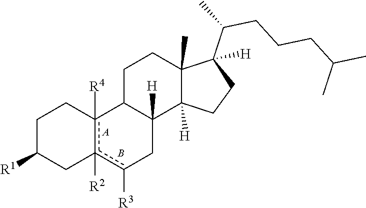

R¹ is OH or OP;
R², when present, is OH or X;
R³ is H, OH, X, CH₂—X, or CH₂-LG;
R⁴, when present, is C₁₋₆ alkyl, C₁₋₆ alkylene-X, or C₁₋₆ alkylene-LG;
X is a halogen; P is an alcohol protecting group;
LG is a leaving group;
each of bond A and bond B is a single or a double bond and only one of bond A and bond B can be a double bond;
with the proviso that:
   at least one X or LG is present; and if LG is present, R¹ is OP;
   if one of R² and R³ is F and the other OH, then the F is ¹⁸F; and
the compound is not:

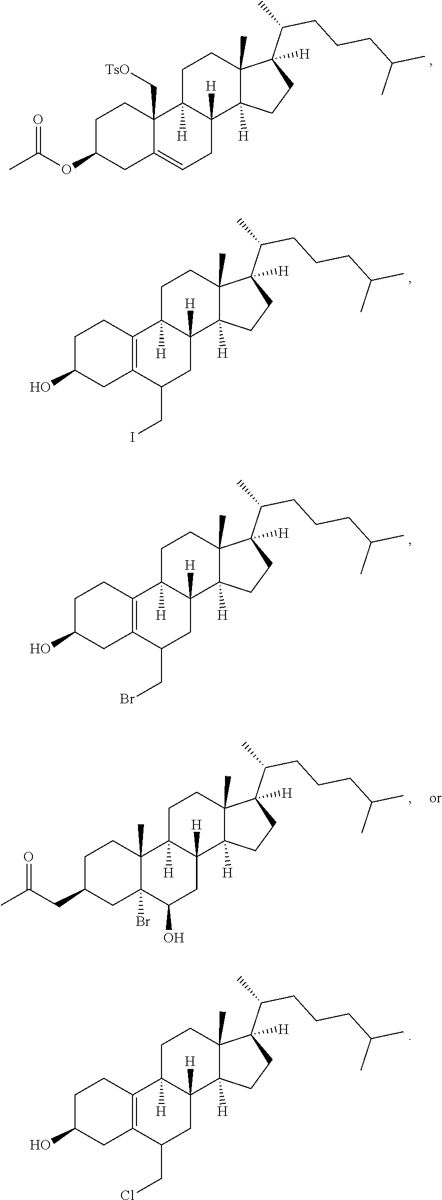

As disclosed herein, X is a halogen. In certain embodiments, X is F or I.

In embodiments, X can be a radioisotope. As used herein, a “radioisotope” refers to an unstable, radioactive isotope that emits excess energy in the form of one or more of α, β, and γ radiation. Examples of common radioisotopes of halogens include, for example, ³⁷Cl, ¹⁸F, ⁷⁷Br, ¹²⁴I, and ¹³¹I. Furthermore, as used herein, a “hot” compound refers to any compound including a radioisotope, whereas a “cold” compound refers to any compound including a stable, non-radioactive isotope. Accordingly, the terms “hot” and “radiolabeled” can be used interchangeably, while the terms “cold” and “non-radiolabeled” can be used interchangeably.

In some cases where X is F, X is specifically ¹⁸F. In some cases where X is I, X is specifically ¹²⁴I or ¹³¹I.

In certain aspects, R¹ is OH. In other aspects, R¹ is OP. In various cases, P is pivaloyl, acetoxy, THP, or MOM. In embodiments, P is THP or MOM.

In certain aspects, R² is X. In other aspects, R² is OH.

In various aspects, R³ is X or CH₂—X. In some embodiments, R³ is CH₂-LG. In embodiments, LG is tosyl, a halogen, mesyl, or triflate. In some embodiments, LG is tosyl or mesyl.

In some aspects, R⁴ is C₁₋₆alkylene-X.

In various cases, A is a double bond. In other cases, B is a double bond. In some cases, each of A and B is a single bond.

In some embodiments, the compound has a structure of Formula (IA):

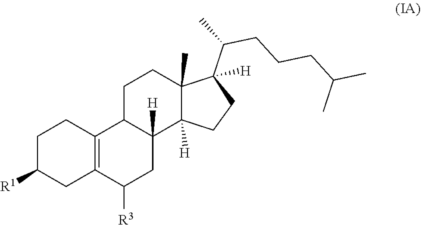

wherein R³ is C₁₋₆ alkylene-X or C₁₋₆ alkylene-LG. In some aspects, R¹ is OP and R³ is CH₂-LG. In some aspects, P is acetoxy and LG is OTs. In other cases, P is MOM or THP and LG is OTs or OMs. In some cases, R³ is CH₂—OTs or CH₂—OMs. In some embodiments, R¹ is OP and R³ is CH₂—X. In some cases, P is pivaloyl and LG is OMs.

In some embodiments, the compound has a structure of Formula (IB):

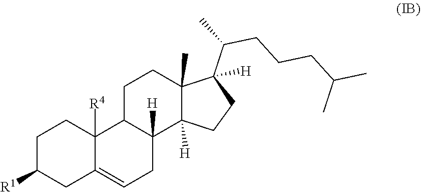

wherein R⁴ is C₁₋₆ alkylene-X or C₁₋₆ alkylene-LG. In some aspects, R¹ is OP and R⁴ is C₁₋₆ alkylene-LG. In some cases, P is acetoxy and LG is OTs. In other cases, P is MOM or THP and LG is OTs or OMs. In some embodiments, R⁴ is CH₂—OTs or CH₂—OMs. In some cases, R¹ is OH and R⁴ is C₁₋₆ alkylene-X. In some cases, R⁴ is CH₂—X.

In some embodiments, the compound has a structure of formula (IC)

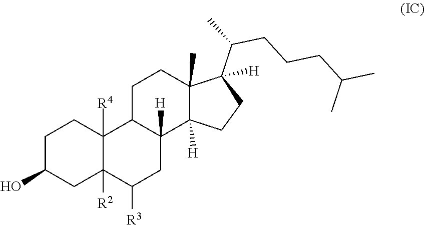

wherein one of R² and R³ is OH and the other is X, and R⁴ is C₁₋₆ alkylene. In some aspects, R⁴ is methyl. In some cases, R² is X and R³ is OH. In other embodiments, R² is OH and R³ is X.

In some embodiments, the disclosure provides compounds having a structure selected from:

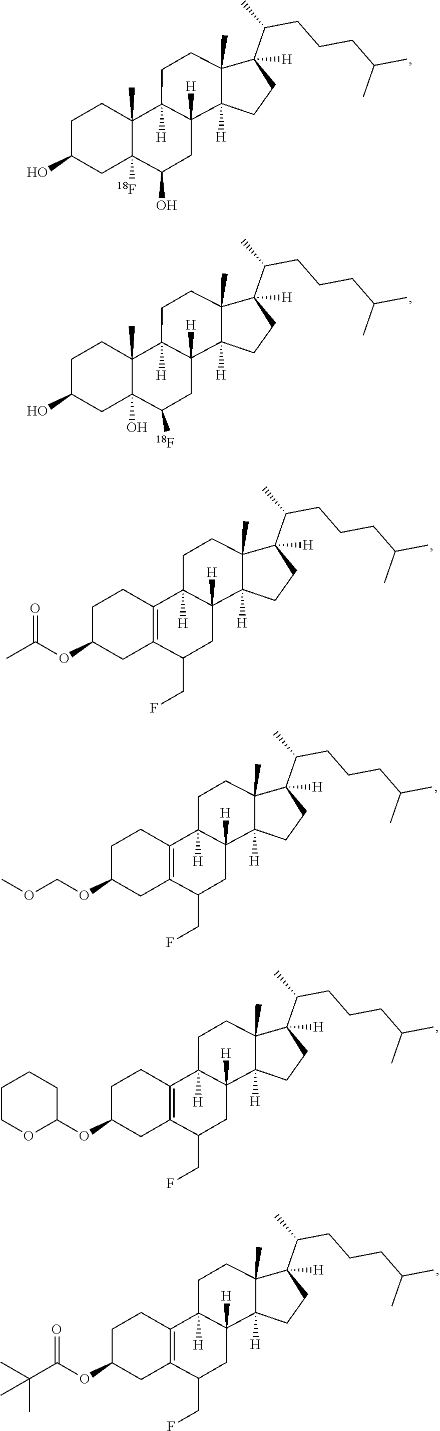

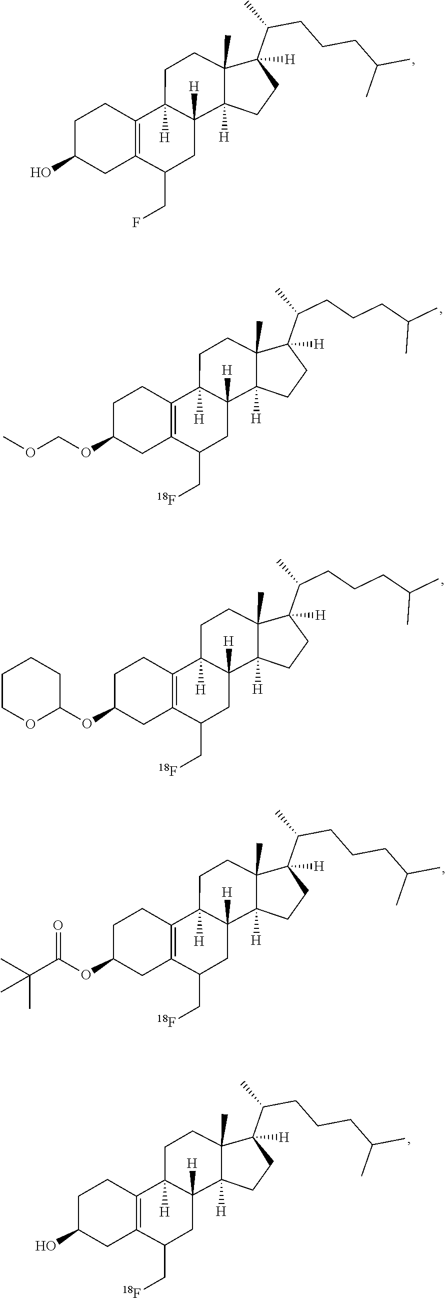

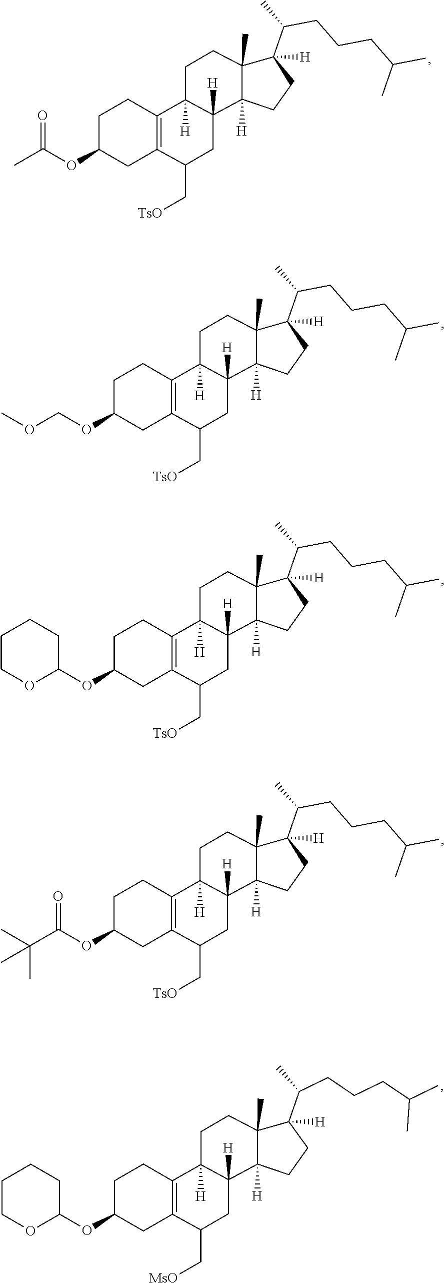

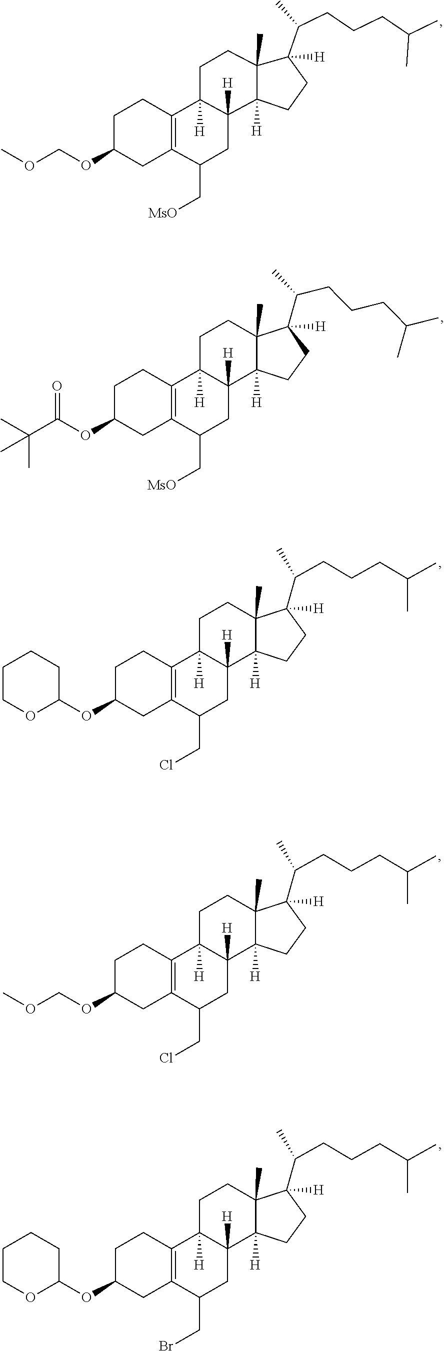

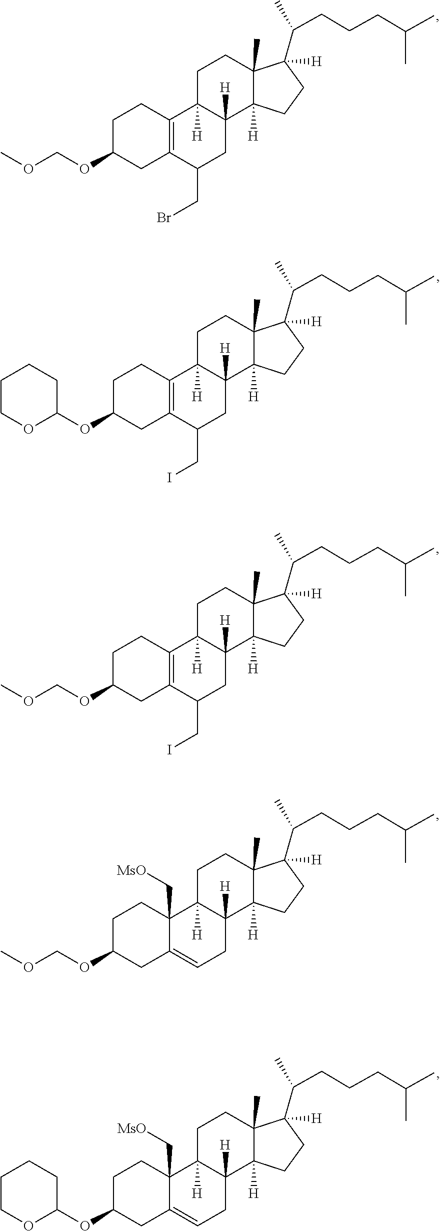

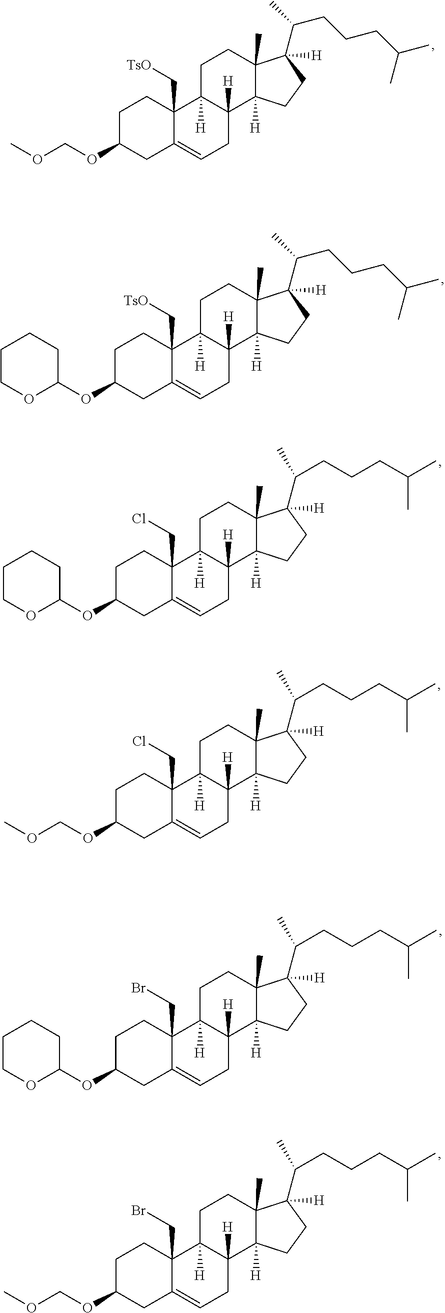

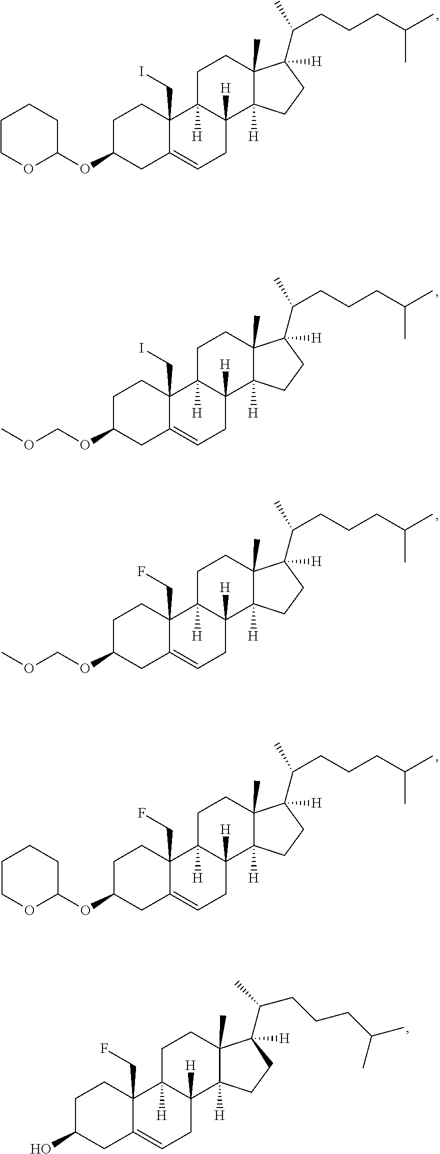

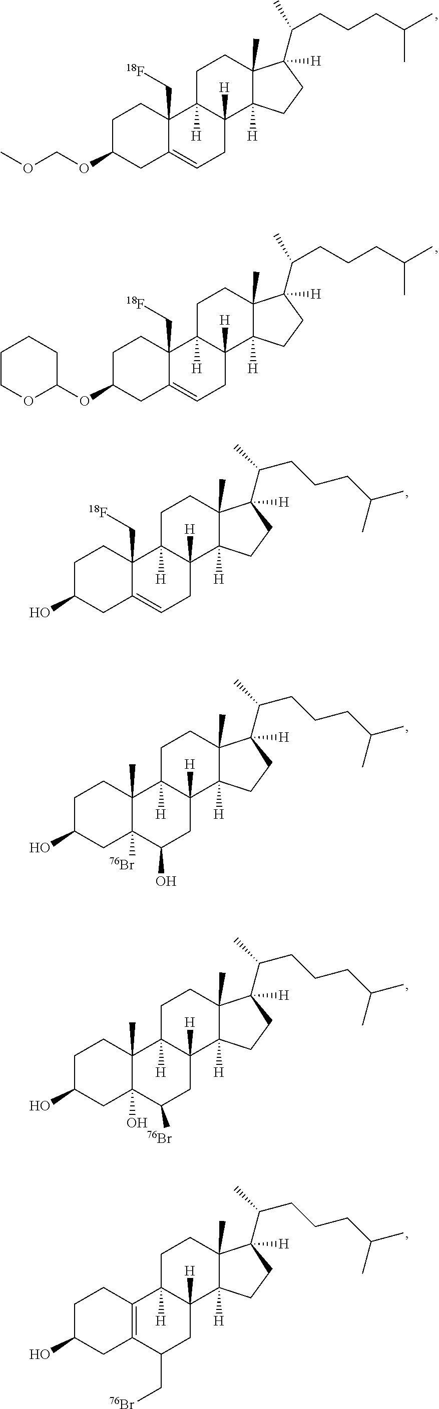

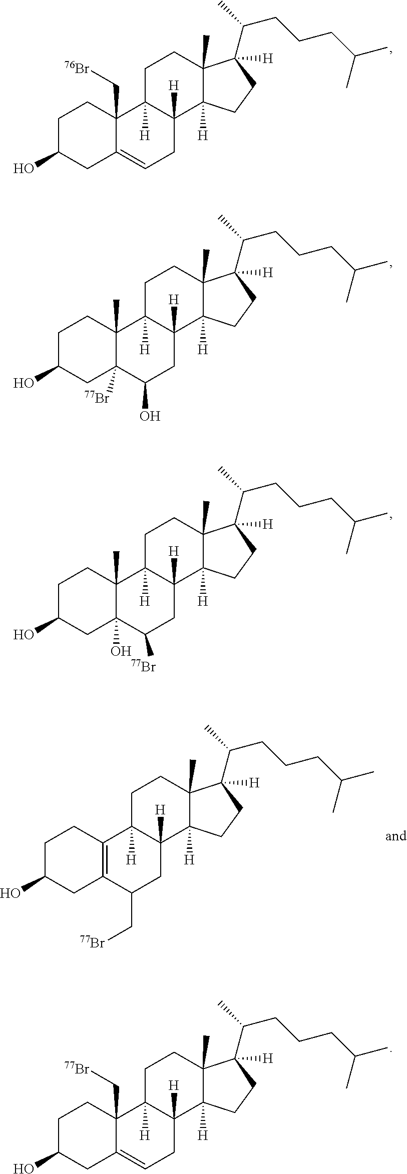

In some aspects, the compound has a structure selected from:

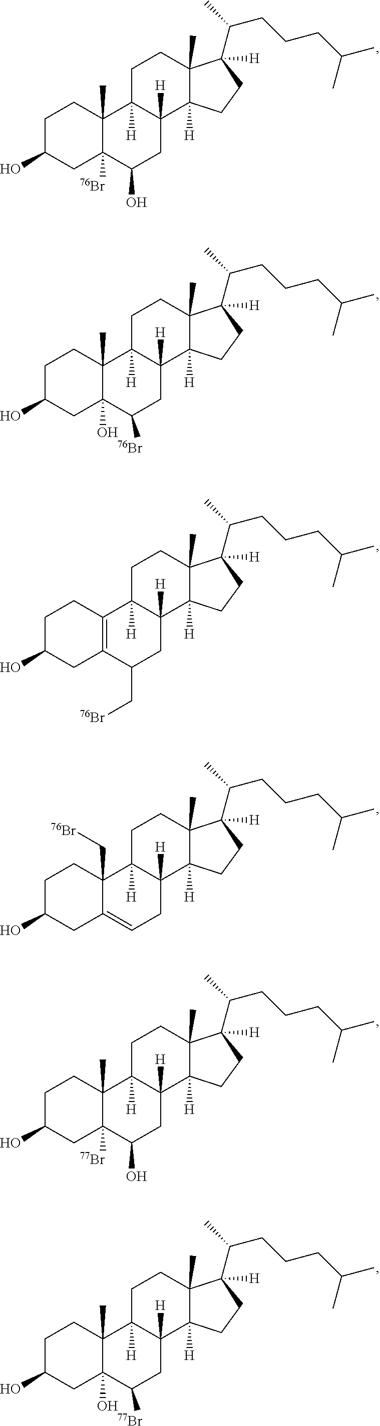

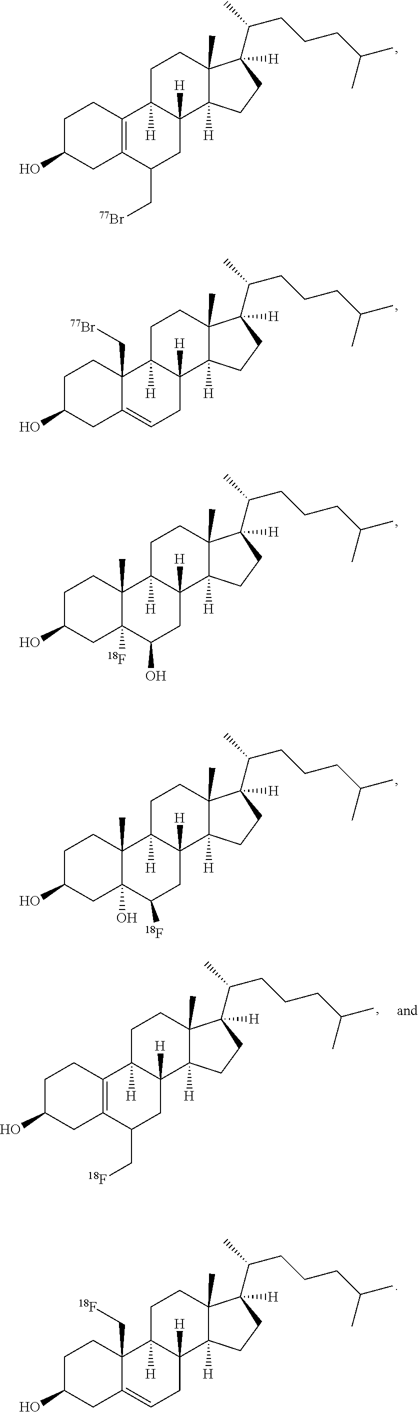

In some aspects, the compound has a structure selected from:

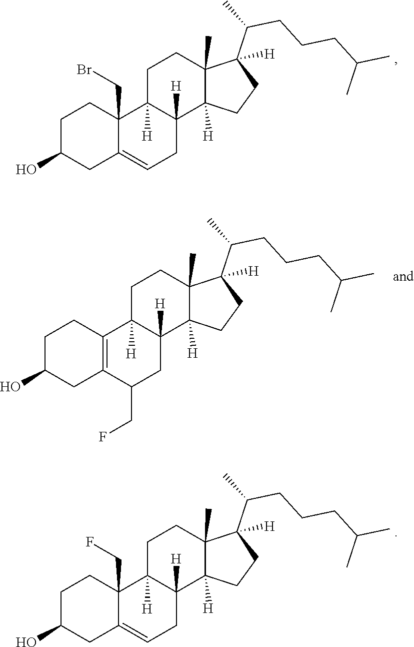

### Methods of Making Radiolabeled Cholesterol Analogues

The disclosure further provides methods of preparing radiolabeled cholesterol analogues.

In embodiments, the disclosure provides a method including admixing a cholesterol epoxide with a metal catalyst and a fluorine-18 source to form a α,β-hydroxy fluoride cholesterol compound, wherein the fluorine-18 source includes H-¹⁸F.

The disclosure further provides a method of preparing a compound having the structure of Formula (II)

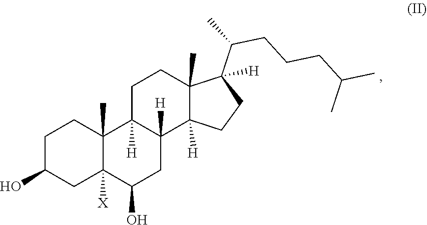

wherein X is ¹⁸F, ⁷⁶Br, or ⁷⁷Br, and the method includes admixing 5,6-epoxycholesterol and a radiolabeled source under conditions sufficient to form the compound of Formula (II).

In embodiments the radiolabeled source can include fluorine-18, bromine-76, or bromine-77.

The fluorine-18 source is not particularly limited. In embodiments, the fluorine-18 source includes H-¹⁸F. Other suitable sources of fluorine-18 for use in the methods described herein include, but are not limited to fluorine-18 salts having counterions such as K, Na, Cs, or transition metals, such as Ag. For example, the fluorine-18 source can include K-¹⁸F, Na-¹⁸F, Cs-¹⁸F, or Ag-¹⁸F.

Without intending to be bound by theory, it is believed the method proceeds under acidic conditions. For example, the method can proceed wherein H-¹⁸F is the both the fluorine-18 source and acid source. In embodiments, the method can include other acids suitable for the reaction, such as HCl, HBr, HI, H₃PO₄, H₂SO₄, or other inorganic acids.

In some cases, the radiolabeled source is present in a substoichiometric amount relative to the epoxide. In embodiments, fluorine-19 can be additionally added as a carrier or diluent in the reaction.

The metal catalyst is not particularly limited. In embodiments the metal catalyst includes a metal such as iron, cobalt, vanadium, copper, ruthenium, indium, nickel, manganese or gallium. Generally, the metal catalyst can include any of the foregoing metals present in a salt or an oxide. Without intending to be bound by theory, metal salts and/or metal oxides are capable of trapping the fluorine-18 source, for example, H-¹⁸F, as a metal fluoride. In embodiments, the metal catalyst includes a metal salt. In various cases, the metal catalyst comprises ferric acetylacetonate. In some cases, the metal catalyst comprises gallium acetylacetonate. Other suitable metal catalysts include, but are not limited to, cobalt acetylacetonate, vanadyl acetylacetonate, cupric acetylacetonate, ruthenium acetylacetonate, indium acetylacetonate, nickel acetylacetonate, or manganese acetylacetonate. In embodiments, the metal catalyst includes a metal oxide. Suitable metal oxides for use as the metal catalyst include, but are not limited to, silver oxide, cupric oxide, cuprous oxide, vanadium pentoxide, iron oxide, ruthenium oxide, indium oxide, nickel oxide, and manganese oxide.

In some embodiments, the method includes admixing the epoxide, for example 5,6-epoxycholesterol, and a fluorine-18 source at a temperature ranging from about 50° C. to about 150° C., about 60° C. to about 140° C., about 70° C. to about 130° C., about 80° C., to about 120° C., about 90° C. to about 110° C., or about 100° C. to about 105° C., for example about 50, 55, 60, 65, 70, 75, 80, 85, 90, 95, 100, 105, 110, 115, 120, 125, 130, 135, 140, 145, or 150° C.

In some embodiments, the admixing step occurs for less than about 1 hour. In embodiments, the admixing step occurs for a period of time ranging from about 5 to about 60 minutes, about 5 to about 45 minutes, about 5 to about 30 minutes, about 10 to about 40 minutes, about 10 to about 25 minutes, about 15 to about 35 minutes, about 15 to about 20 minutes, about 20 to about 30 minutes, about 30 to about 60 minutes, about 30 to about 45 minutes, about 45 to about 60 minutes, or about 40 to about 50 minutes, for example about 5, 10, 15, 20, 25, 30, 35, 40, 45, 50, 55, or 60 minutes.

Without intended to be bound by theory, the admixing step is preferably no longer than about 1 hour due to the half-life of ¹⁸F. The half-life of ¹⁸F is approximately 110 minutes. Accordingly, in order for the fluorine-18 source used in the disclosed method to be prepared, admixed and reacted with the epoxide, prepared for administration to a subject (inclusive of any purification and processing steps), administered to the subject, and subsequently imaged while still have measurable radioactivity, the methods described herein preferably have admixing steps of no longer than about 60 minutes.

In embodiments, the disclosure provides a method comprising admixing cholesterol and an acyl chloride (e.g. pivaloyl chloride or other suitable acyl chloride protecting group, e.g., benzoyl chloride or acetyl chloride) to form cholest-5-en-3-acylate (e.g., cholest-5-en-3-pivaloate). The admixing of cholesterol and the acyl chloride (e.g. pivalyol chloride) can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the admixing of cholesterol and the acyl chloride (e.g., pivaloyl chloride) occurs in dichloromethane. The admixture of cholesterol and the acyl chloride (e.g., pivaloyl chloride) can further include reagents such as, but not limited to, triethylamine (TEA or Et₃N) and/or dimethylaminopyridine (DMAP). The admixing of cholesterol and the acyl chloride (e.g., pivaloyl chloride) can take place for a period of time ranging from about 1 hour to about 48 hours, about 5 hours to about 36 hours, about 10 hours to about 24 hours, or about 15 hours to about 20 hours, for example about 1, 2, 3, 4, 5, 7, 10, 12, 15, 17, 18, 20, 22, 24, 26, 30, 32, 35, 37, 40, 42, 45, or 48 hours. The admixing can be carried out at a temperature ranging from about 0° C. to about 35° C., about 5° C. to about 30° C., about 10° C. to about 25° C., or about 15° C. to about 20° C., for example about 0, 2, 5, 7, 10, 12, 15, 17, 20, 22, 25, 27, or 30° C.

In embodiments, the method further comprises reacting cholest-5-en-3-acylate (e.g., cholest-5-en-3-pivaloate) with N-bromoacetamide to form a 5-bromocholestan-6-hydroxy-3-acylate (e.g., 5-bromocholestan-6-hydroxy-3-pivaloate). The reacting of cholest-5-en-3-acylate (e.g. cholest-5-en-3-pivaloate) and N-bromoacetamide can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the reacting of cholest-5-en-3-acylate (e.g., cholest-5-en-3-pivaloate) and N-bromoacetamide occurs in dioxane (e.g., 1,4-dioxane). The reaction mixture of cholest-5-en-3-acylate (e.g., cholest-5-en-3-pivaloate) and N-bromoacetamide can further include reagents such as, but not limited to, a strong acid (e.g., perchloric acid) and/or a quenching agent (e.g., sodium thiosulfate). In some cases, the quenching agent is provided in an aqueous solution, for example, a 10% sodium thiosulfate aqueous solution. The reacting of cholest-5-en-3-acylate (e.g., cholest-5-en-3-pivaloate) and N-bromoacetamide can take place for a period of time ranging from about 5 minutes to about 2 hours, about 10 minutes to about 1 hour, about 20 minutes to about 40 minutes, or about 25 minutes to about 35 minutes, for example about 5, 10, 15, 20, 25, 30, 35, 40, 45, 50, 55, 60, 70, 80, 90, 100, 110 or 120 minutes. The reacting can be carried out at a temperature ranging from about 0° C. to about 35° C., about 5° C. to about 30° C., about 10° C. to about 25° C., or about 15° C. to about 20° C., for example about 0, 2, 5, 7, 10, 12, 15, 17, 20, 22, 25, 27, or 30° C.

In embodiments, the method further comprises reacting the 5-bromocholestan-6-hydroxy-3-acylate (e.g., 5-bromocholestan-6-hydroxy-3-pivolate) with lead tetraacetate to form a 5-bromocholestan-6(19)-oxo-3-acylate (e.g., 5-bromocholestan-6(19)-oxo-3-pivolate). The reacting of 5-bromocholestan-6-hydroxy-3-acylate (e.g., 5-bromocholestan-6-hydroxy-3-pivolate) and lead tetraacetate can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the reacting of 5-bromocholestan-6-hydroxy-3-acylate (e.g., 5-bromocholestan-6-hydroxy-3-pivolate) and lead tetraacetate occurs in cyclohexane. The reaction mixture of 5-bromocholestan-6-hydroxy-3-acylate (e.g., 5-bromocholestan-6-hydroxy-3-pivolate) and lead tetraacetate can further include reagents such as, but not limited to, iodine. The reacting of 5-bromocholestan-6-hydroxy-3-acylate (e.g., 5-bromocholestan-6-hydroxy-3-pivolate) and lead tetraacetate can take place for a period of time ranging from about 5 minutes to about 3 hours, about 20 minutes to about 2 hours, or about 30 minutes to about 1 hour, for example about 5, 10, 15, 20, 25, 30, 35, 40, 45, 50, 55, 60, 70, 80, 90, 100, 110, 120, 130, 140, 150, 160, 170, or 180 minutes. The reacting can be carried out at a temperature ranging from about 15° C. to about 100° C., about 30° C. to about 90° C., about 40° C. to about 80° C., or about 50° C. to about 70° C., for example about 15, 20, 25, 30, 35, 40, 45, 50, 55, 60 65, 70, 75, 80, 85, 90, 95, or 100° C.

In embodiments, the method further comprises reacting 5-bromocholestan-6(19)-oxo-3-acylate (e.g., 5-bromocholestan-6(19)-oxo-3-pivolate) with activated zinc to form a cholest-5-en-19-hydroxy-3-acylate (e.g., cholest-5-en-19-hydroxy-3-pivaloate). As used herein, “activated” means that the zinc, which can be initially present in the form of an unreactive zinc powder, has been subjected to conditions sufficient to make it into a reactive compound for use in the synthesis reaction. For example, in some cases, the unreactive zinc powder is activated under heat and vacuum. The reacting of 5-bromocholestan-6(19)-oxo-3-acylate (e.g., 5-bromocholestan-6(19)-oxo-3-pivaloate) and activated zinc can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the reacting of 5-bromocholestan-6(19)-oxo-3-acylate (e.g., 5-bromocholestan-6(19)-oxo-3-pivaloate) and activated zinc occurs in isopropanol. The reaction mixture of 5-bromocholestan-6(19)-oxo-3-acylate (e.g., 5-bromocholestan-6(19)-oxo-3-pivaloate) and activated zinc can further include reagents such as, but not limited to, glacial acetic acid. The reacting of 5-bromocholestan-6(19)-oxo-3-acylate (e.g., 5-bromocholestan-6(19)-oxo-3-pivaloate) and activated zinc can take place for a period of time ranging from about 1 hour to about 20 hours, about 5 hours to about 18 hours, or about 10 hours to about 15 hours, for example about 1, 2, 3, 4, 5, 6, 7, 8, 9, 10, 11, 12, 13, 14, 15, 16, 17, 18, 19 or 20 hours. The reacting can be carried out at a temperature ranging from about 15° C. to about 100° C., about 30° C. to about 90° C., about 40° C. to about 80° C., or about 50° C. to about 70° C., for example about 15, 20, 25, 30, 35, 40, 45, 50, 55, 60 65, 70, 75, 80, 85, 90, 95, or 100° C. In some cases, the reaction is carried out at two or more different temperatures for two or more different periods of time. For example, in some cases, the reaction includes stirring for about 30 minutes at a temperature of 90° C., followed by stirring for about 18 hours at ambient room temperature.

In embodiments, the method further comprises reacting the cholest-5-en-19-hydroxy-3-acylate (e.g., cholest-5-en-19-hydroxy-3-pivaloate) with mesyl chloride then potassium acetate to form (3S,5R,10S,13R,17R)-6-hydroxy-13-methyl-17-((R)-6-methylheptan-2-yl)tetradecahydro-6H-5,10-methanocyclopenta[a]phenanthren-3-yl acylate (e.g., (3S,5R,10S,13R,17R)-6-hydroxy-13-methyl-17-((R)-6-methylheptan-2-yl)tetradecahydro-6H-5,10-methanocyclopenta[a]phenanthren-3-yl pivaloate). The reacting of cholest-5-en-19-hydroxy-3-acylate (e.g., cholest-5-en-19-hydroxy-3-pivaloate) and mesyl chloride can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the reacting of cholest-5-en-19-hydroxy-3-acylate (e.g., cholest-5-en-19-hydroxy-3-pivaloate) and mesyl chloride occurs in pyridine. The reaction mixture of cholest-5-en-19-hydroxy-3-acylate (e.g., cholest-5-en-19-hydroxy-3-pivaloate) and mesyl chloride can further include reagents such as, but not limited to, methanesulfonyl chloride, and a quenching agent (e.g. cold water). The reacting of cholest-5-en-19-hydroxy-3-acylate (e.g., cholest-5-en-19-hydroxy-3-pivaloate) and mesyl chloride can take place for a period of time ranging from about 1 hour to about 5 hours, about 2 hours to about 4 hours, or about 1 hour to about 3 hours, for example about 1, 2, 3, 4, or 5 hours. The reacting can be carried out at a temperature ranging from about 0° C. to about 30° C., about 5° C. to about 25° C., about 10° C. to about 20° C., or about 15° C. to about 20° C., for example about 0, 1, 2, 3, 4, 5, 7, 10, 12, 15, 18, 20, 22, 25, 27, or 30° C. The product of the reaction between cholest-5-en-19-hydroxy-3-acylate (e.g., cholest-5-en-19-hydroxy-3-pivaloate) and mesyl chloride can then be reacted with potassium acetate. The reacting of the product with potassium acetate can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the reacting of the product and potassium acetate occurs in 3-pentanone. The reaction mixture of the product and potassium acetate can further include reagents such as, but not limited to, water. The reacting of the product with potassium acetate can take place for a period of time ranging from about 1 hour to about 48 hours, about 5 hours to about 36 hours, about 10 hours to about 24 hours, or about 15 hours to about 20 hours, for example about 1, 2, 3, 4, 5, 7, 10, 12, 15, 17, 18, 20, 22, 24, 26, 30, 32, 35, 37, 40, 42, 45, or 48 hours. The reacting can be carried out at a temperature ranging from about 15° C. to about 150° C., about 30° C. to about 120° C., about 50° C. to about 100° C., or about 75° C. to about 90° C., for example about 15, 20, 25, 30, 35, 40, 45, 50, 55, 60, 65, 70, 75, 80, 85, 90, 95, 100, 105, 110, 115, 120, 125, 130, 135, 140, 145, or 150° C.

In embodiments, the method further comprises reacting (3S,5R,10S,13R,17R)-6-hydroxy-13-methyl-17-((R)-6-methylheptan-2-yl)tetradecahydro-6H-5,10-methanocyclopenta[a]phenanthren-3-yl acylate (e.g., (3S,5R,10S,13R,17R)-6-hydroxy-13-methyl-17-((R)-6-methylheptan-2-yl)tetradecahydro-6H-5,10-methanocyclopenta[a]phenanthren-3-yl pivaloate) with boron trifluoride and methanesulfonic acid to form 6-methyl(methanesulfonyl)-19-norcholest-5(10)-en-3-yl acylate (e.g., 6-methyl(methanesulfonyl)-19-norcholest-5(10)-en-3-yl pivaloate). The reacting of (3S,5R,10S,13R,17R)-6-hydroxy-13-methyl-17-((R)-6-methylheptan-2-yl)tetradecahydro-6H-5,10-methanocyclopenta[a]phenanthren-3-yl acylate (e.g., (3S,5R,10S,13R,17R)-6-hydroxy-13-methyl-17-((R)-6-methylheptan-2-yl)tetradecahydro-6H-5,10-methanocyclopenta[a]phenanthren-3-yl pivaloate) with boron trifluoride and methanesulfonic acid can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the reacting occurs in dichloromethane. The reaction can further be carried out under argon gas. The reacting can take place for a period of time ranging from about 1 hour to about 5 hours, about 2 hours to about 4 hours, or about 1 hour to about 4 hours, for example about 1, 2, 3, 4, or 5 hours. The reacting can be carried out at a temperature ranging from about 0° C. to about 30° C., about 5° C. to about 25° C., about 10° C. to about 20° C., or about 15° C. to about 20° C., for example about 0, 1, 2, 3, 4, 5, 7, 10, 12, 15, 18, 20, 22, 25, 27, or 30° C.

In some cases, the method further comprises reacting 6-methyl(methanesulfonyl)-19-norcholest-5(10)-en-3-yl acylate (e.g., 6-methyl(methanesulfonyl)-19-norcholest-5(10)-en-3-yl pivaloate) with an ¹⁸F source then treating with a strong base to form ¹⁸F-FNP-59. In some cases, the strong base comprises potassium hydroxide. In embodiments, the ¹⁸F source is prepared using a cyclotron, according to methods known in the art. Nonlimiting examples of the ¹⁸F source include NBu₄[¹⁸F]F and NEt₄[¹⁸F]F. The ¹⁸F source can then be delivered to the reaction vessel with tetraethylammonium bicarbonate or tetrabutylammonium bicarbonate in water. The reaction vessel can further include a reagent such as, but not limited to, acetonitrile. The ¹⁸F source can be azeotropically dried under various conditions, such as heat (e.g. greater than 50, 75, 80, or 90° C. and/or up to 75, 85, 95, or 100° C.), pressure (e.g. vacuum), and/or atmosphere (e.g. argon gas). To the reaction vessel containing the azeotropically dried ¹⁸F source, 6-methyl(methanesulfonyl)-19-norcholest-5(10)-en-3-yl acylate (e.g., 6-methyl(methanesulfonyl)-19-norcholest-5(10)-en-3-yl pivaloate) can be added. 6-Methyl(methanesulfonyl)-19-norcholest-5(10)-en-3-yl acylate (e.g., 6-methyl(methanesulfonyl)-19-norcholest-5(10)-en-3-yl pivaloate) can be present in an organic solvent, such as, for example, acetonitrile. The reacting can take place for a period of time ranging from about 5 minutes to about 60 minutes, about 10 minutes to about 45 minutes, or about 15 minutes to about 35 minutes, for example, 5, 10, 15, 20, 25, 30, 35, 40, 45, 50, 55 or 60 minutes. The reacting can be carried out at a temperature ranging from about 15° C. to about 150° C., about 30° C. to about 120° C., about 50° C. to about 100° C., or about 75° C. to about 90° C., for example about 15, 20, 25, 30, 35, 40, 45, 50, 55, 60, 65, 70, 75, 80, 85, 90, 95, 100, 105, 110, 115, 120, 125, 130, 135, 140, 145, or 150° C. Subsequently, a strong base, such as potassium hydroxide, can be added, and reacted for a period of time and at a temperature as provided for the reaction of 6-methyl(methanesulfonyl)-19-norcholest-5(10)-en-3-yl acylate (e.g., 6-methyl(methanesulfonyl)-19-norcholest-5(10)-en-3-yl pivaloate) with the ¹⁸F source, above.

In some cases, the method further comprises reacting 6-methyl(methanesulfonyl)-19-norcholest-5(10)-en-3-yl acylate (e.g., 6-methyl(methanesulfonyl)-19-norcholest-5(10)-en-3-yl pivaloate) with tetrabutylammonium fluoride (TBAF) to form fluorinated NP-59 (FNP-59).

In some cases, the TBAF can be present in the reaction mixture as TBAF bis(pinacol). The reacting of 6-methyl(methanesulfonyl)-19-norcholest-5(10)-en-3-yl acylate (e.g., 6-methyl(methanesulfonyl)-19-norcholest-5(10)-en-3-yl pivaloate) and TBAF can take place in a suitable organic solvent, including, but not limited to, dichloromethane (DCM), dioxane, cyclohexane, isopropanol, acetone, pyridine, 3-pentanone, acetonitrile (MeCN or ACN), or ethanol. In some cases, the reacting occurs in acetonitrile. The reacting can take place for a period of time ranging from about 1 hour to about 5 hours, about 2 hours to about 4 hours, or about 1 hour to about 4 hours, for example about 1, 2, 3, 4, or 5 hours. The reacting can be carried out at a temperature ranging from about 15° C. to about 100° C., about 30° C. to about 90° C., about 40° C. to about 80° C., or about 50° C. to about 70° C., for example about 15, 20, 25, 30, 35, 40, 45, 50, 55, 60 65, 70, 75, 80, 85, 90, 95, or 100° C.

### Use of Cholesterol Analogues

The disclosure further provides methods of using the compounds described herein. In particular, the disclosure provides methods including administering to a subject a compound as described herein and subjecting the subject to an imaging modality.

The manner of administration of the compound is not particularly limited. For example, in embodiments, the compound can be administered intravenously or orally. The manner of administration and dose thereof would be within the purview of the doctor, nurse, or radiologist trained to administer these compounds.

In embodiments, the imaging modality can be selected from positron emission tomography (PET), positron emission tomography/computed tomography (PET/CT), positron emission tomography/magnetic resonance imaging (PET/MRI), planar gamma camera imaging, single-photon emission computerized tomography (SPECT), and/or single-photon emission computerized tomography/computed tomography (SPECT/CT).

Generally, it is envisaged that the compounds disclosed herein include a radioisotope when the subject is subjected to the imaging modality. However, in particular embodiments, the compound can include a non-radiolabeled compound, that is, a compound including, for example, ¹⁹F, and still remain suitable for imaging. For example, PET/MRI can be used to image cold compounds, such as those including ¹⁹F or ¹²⁷I.

In embodiments, the subject suffers or is suspected of suffering from Cushing's syndrome, primary aldosteronism, hyperandrogenism, adenoma, gonadal disease, pheochromocytoma, an atherosclerotic disease, a disorder of cholesterol metabolism and distribution, or ectopic cholesterol production. In some cases, the adenoma is an adrenal adenoma. In some cases the adenoma is a non-adrenal adenoma. In some cases, the atherosclerotic disease comprises vulnerable plaque. In some cases, the patient has vulnerable plaque and the imaging step identifies the vulnerable plaque. In some cases, the gonadal disease comprises tumors of the ovaries or testis. In some cases, the subject suffers from or is suspected of suffering from an Akt-associated disorder. In some cases, the disorder of cholesterol metabolism and distribution involves the circulating LDL/HDL cholesterol pool.

In embodiments, the use of the compound described herein can include locating sites of ectopic cholesterol production, as well as imaging normal and pathologic cholesterol metabolism in, for example, gonadal tissue with and without steroid production. In embodiments, the compound can be used to image cholesterol metabolism in the cardiovascular system. In some embodiments, the compound can be used to image non-adrenal adenomas such as breast cancer.

In embodiments, the subject is subjected to the imaging modality at a point in time ranging from about 0.5 hours to 7 days after of the compound. The time at which the subject is subjected to the imaging modality is dependent on the isotope of the halogen used in the cholesterol analogue. For example, due to the short half-life of ¹⁸F, when the compound is radiofluorinated, the subject can be subjected to the imaging modality at a point in time ranging from about 0.5 hours to about 5 hours, about 0.6 hours to about 4.5 hours, about 0.7 hours to about 4 hours, about 0.8 hours to about 3.5 hours, about 0.9 hours to about 3 hours, or about 1 hour to about 2 hours, for example at about 0.5, 0.6, 0.7, 0.8, 0.9, 1, 1.5, 2, 2.5, 3, 3.5, 4, 4.5, or 5 hours after administration of the compound. Due to the half-life of ¹²⁴I, for example, having a t_{1/2}=4.2 days, when the compound is radioiodonated, the subject can be subjected to the imaging modality at a point in time ranging from about 0.5 hours to about 7 days, from about 5 hours to about 5 days, from about 12 hours to about 3 days, or from about 1 day to about 2 days, for example at about 0.5 hours, about 1 hour, about 2 hours, about 5 hours, about 7 hours, about 12 hours, about 1 day, about 2 days, about 3 days, about 4 days, about 5 days, about 6 days, or about 7 days after administration of the compound.

In some cases, the method further comprises administering to the subject a drug or steroid prior to the administration of the compound as described herein. For example, the subject can be administered a steroid such as dexamethasone, prednisone, solumedrol, or the like. In embodiments, the drug and/or steroid is administered concurrently with the compound described herein. In embodiments, the drug and/or steroid is administered prior to administration of the compound described herein, for example, about 3 to about 7 days prior to administration of the compound. The drug and/or steroid can be used to promote or suppress biological cholesterol metabolism in the tissue of interest, or, alternatively, in background tissue surrounding the tissue of interest.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

### Methods and Materials

All commercial products were used as received and reagents were stored under ambient conditions unless otherwise stated. The manipulation of solid reagents was conducted on the benchtop unless otherwise stated. Reactions were conducted under an ambient atmosphere unless otherwise stated. Reaction vessels were sealed with a septum. Reactions conducted at elevated temperatures were heated with an oil bath. Temperatures were regulated using an external thermocouple. For TLC analysis, R_{F} values are reported based on normal phase silica plates with fluorescent indicator and I₂ staining.

### Instrumental Information

NMR spectra were obtained on a Varian MR400 (400.53 MHz for ¹H; 100.13 MHz for ¹³C; 376.87 MHz for ¹⁹F) spectrometer. All ¹³C NMR data presented are proton-decoupled ¹³C NMR spectra, unless noted otherwise. ¹H and ¹³C NMR chemical shifts (6) are reported in parts per million (ppm) relative to TMS with the residual solvent peak used as an internal reference. ¹H and ¹⁹F NMR multiplicities are reported as follows: singlet (s), doublet (d), triplet (t), quartet (q), and multiplet (m). High performance liquid chromatography (HPLC) was performed using a Shimadzu LC-2010A HT system equipped with a Bioscan B-FC-1000 radiation detector. Radio-TLC analyses were performed using a Bioscan AR 2000 Radio-TLC scanner with EMD Millipore TLC silica gel 60 plates (3.0 cm wide×6.5 cm long).

### Example 1—Synthesis of Fluorinated NP-59 (FMNC)

The scheme of the synthesis of (3S,8S,9S,13R,14S,17R)-6-(fluoromethyl)-13-methyl-17-((R)-6-methylheptan-2-yl)-2,3,4,6,7,8,9,11,12,13,14,15,16,17-tetradecahydro-1H-cyclopenta[a]phenanthren-3-ol (“FMNC”; compound 4) starting from NP-59 is depicted below:

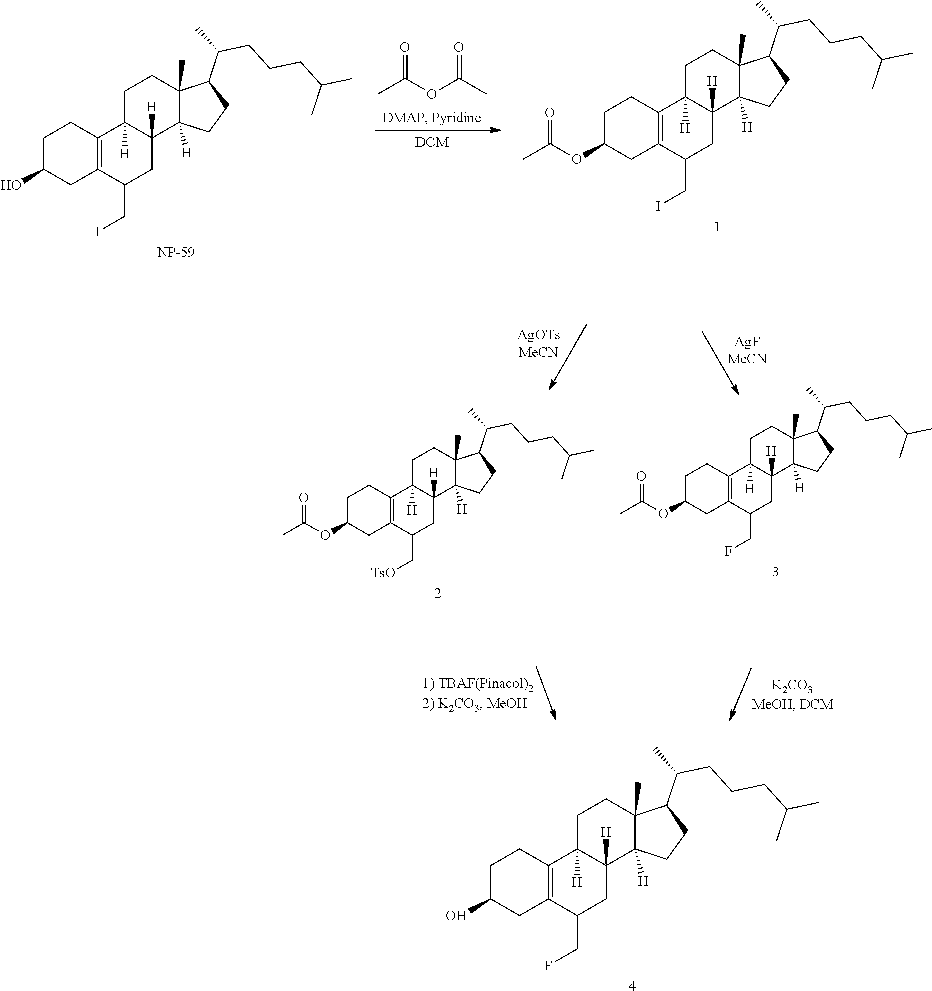

The synthesis of FMNC as described below begins with NP-59 (Dalton Pharma Services). Unlike the synthesis of other halogen analogues of NP-59, it was unexpectedly found that the fluorine analogue could not be prepared by halex exchange with NP-59. It was found that the hydroxyl of NP-59 had to first be protected before fluorination could occur.

### Synthesis of Compound 1

The synthesis of (3S,8S,9S,13R,14S,17R)-6-(iodomethyl)-13-methyl-17-((R)-6-methylheptan-2-yl)-2,3,4,6,7,8,9,11,12,13,14,15,16,17-tetradecahydro-1H-cyclopenta[a]phenanthren-3-yl acetate (“compound 1”) proceeded as follows:

NP-59 (0.1327 g, 0.259 mmol) was added to a flame dried flask and dissolved in DCM (2.5 mL). To this solution DMAP (0.0032 g, 0.26 mmol), pyridine (0.0409 mL, 0.517 mmol) were added and the solution cooled to 0° C. Acetic anhydride (0.049 mL, 0.517 mmol) was added and the solution was allowed to come to room temperature. After 18 h the reaction was dried unto silica gel and purified by flash chromatography (10% ethyl acetate in hexanes) to yield 0.1356 g (94% yield) of the product.

The proton NMR spectrum of compound 1 was as follows: ¹H NMR (400 MHz, CDCl₃) δ 4.95 (m, 1H), 3.40 (m, 1H), 3.02 (t, J=10.5, 1H), 2.01 (s, 3H), 0.93 (d, J=6.4, 3H), 0.84 (d, J=6.6, 6H), 0.67 (s, 3H).

### Synthesis of Compound 2

The synthesis of (3S,8S,9S,13R,14S,17R)-13-methyl-17-((R)-6-methylheptan-2-yl)-6-((tosyloxy)methyl)-2,3,4,6,7,8,9,11,12,13,14,15,16,17-tetradecahydro-1H-cyclopenta[a]phenanthren-3-yl acetate (“compound 2”), proceeded as follows:

Compound 1 (0.080 g, 0.195 mmol) was dissolved in acetonitrile (4 mL). To the solution AgOTs (0.0600, 0.215 mmol) was added. The mixture was stirred and refluxed overnight. The reaction mixture was filtered through a sintered glass funnel to remove AgI. The filtrate was loaded onto florisil and purified with a hexanes ethyl acetate gradient. The product was isolated as an off white solid (0.0333 g, 29% yield).

The proton NMR spectrum of compound 2 was as follows: ¹H NMR (400 MHz, CDCl₃) δ 7.79 (d, J=8.2, 2H), 7.34 (d, J=8.2, 2H), 4.93 (m, 1H), 4.04 (m, 1H), 3.84 (t, J=9.7, 1H), 2.44 (s, 3H), 2.03 (s, 3H), 0.89 (br, 3H), 0.86 (d, J=6.6, 6H), 0.55 (s, 3H).

### Synthesis of Compound 3

The synthesis of (3S,8S,9S,13R,14S,17R)-6-(fluoromethyl)-13-methyl-17-((R)-6-methylheptan-2-yl)-2,3,4,6,7,8,9,11,12,13,14,15,16,17-tetradecahydro-1H-cyclopenta[a]phenanthren-3-yl acetate (“compound 3”), proceeded as follows:

Compound 1 (0.055 g, 0.0992 mmol) was dissolved in acetonitrile (5.5 mL). To the solution AgF (0.050, 0.397 mmol) was added. The mixture was stirred and refluxed for 30 min. The reaction mixture was quenched with brine (15 mL) filtered thru a sintered glass funnel to remove AgI. The filtrate was isolated and utilized in the following step directly.

Synthesis of FMNC from Compound 3

The synthesis of FMNC, 4, from compound 3, proceeded as follows:

Compound 3 was dissolved in a 1:1 mixture of DCM and methanol (1 mL). Potassium carbonate (K₂CO₃) was added and the reaction was stirred overnight. The product was filtered to remove remaining K₂CO₃ and any solids. Deprotection was complete.

The fluorine NMR spectrum of compound 4 was as follows: ¹⁹F NMR (376 MHz, CDC₃) δ−218.

Synthesis of FMNC from Compound 2

The synthesis of FMNC, 4, from compound 2, proceeded as follows:

Compound 2 (0.0280 g, 0.047 mmol) was dissolved in MeCN (1 mL). TBAF(Pin)₂ (0.0470 g, 0.093 mmol) was added and the reaction was heated at 70° C. for 2 h. The reaction was cooled and ether and water were added to quench the reaction. After extraction the material was deprotected by dissolving the material in a 1:1 mixture of DCM and methanol (1 mL). Potassium carbonate (K₂CO₃) was added and the reaction was stirred overnight. The product was filtered to remove remaining K₂CO₃ and any solids. Deprotection was complete.

The proton NMR spectrum of compound 4 was as follows: ¹H NMR (400 MHz, CDCl3) δ 5.1-4.6 (m, 3H), 0.92 (br, 3H), 0.86 (d, J=6.6, 6H), 0.68 (s, 3H). The fluorine NMR spectrum of compound 4 was as follows: ¹⁹F NMR (376 MHz, CDCl3) δ −218.

### Example 2—Synthesis of 19-Fluoro-Cholesterol

The scheme of the synthesis of (3S,10S,13R,17R)-10-(fluoromethyl)-13-methyl-17-((R)-6-methylheptan-2-yl)-2,3,4,7,8,9,10,11,12,13,14,15,16,17-tetradecahydro-1H-cyclopenta[a]phenanthren-3-ol (“19-fluoro-cholesterol”) is shown below:

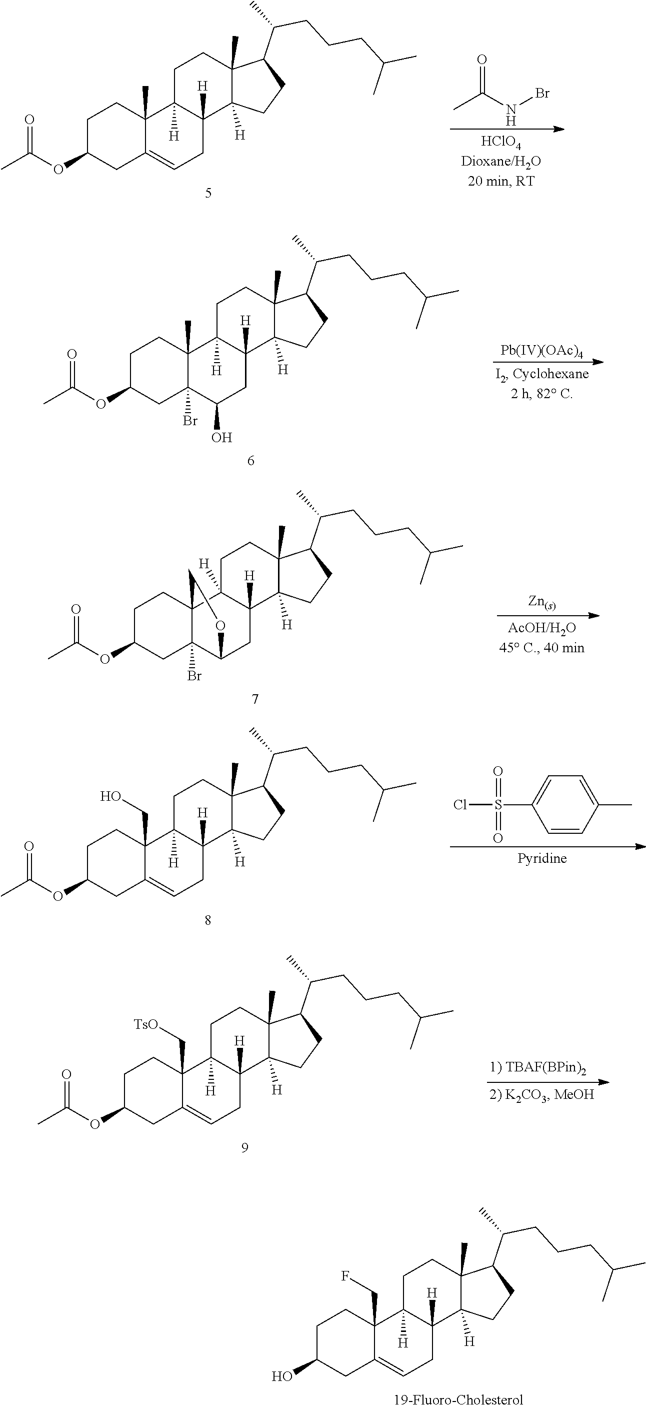

### Synthesis of Compound 5

The synthesis of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl acetate (3-acetoxy-5-cholestene, “compound 5”) proceeded as follows:

Cholesterol (2 g, 5.18 mmol) was dissolved in dichloromethane (40 mL) while stirring. Pyridine (0.84 mL, 10.36 mmol) was added. To this mixture, acetic anhydride (0.98 mL, 10.36 mmol) was added dropwise. The reaction was stirred for 10 hours, before being dried under vacuum. The product was purified by flash chromatography (10 g, 1:9 EtOAc:hexane) to yield a waxy white solid (1.7460 g, 78.6%).

The TLC analysis gave an R_{f}=0.45 in 1:10 EtOAc:Hexane, and the NMR spectrum matched literature reports.

### Synthesis of Compound 6

The synthesis of (3S,5R,6R,8S,9S,10R,13R,14S,17R)-5-bromo-6-hydroxy-10,13-dimethyl-17-((R)-6-methylheptan-2-yl)hexadecahydro-1H-cyclopenta[a]phenanthren-3-yl acetate (3-acetoxy-5-bromo-6-cholestane, “compound 6”) proceeded as follows:

Compound 5 (25 g, 58.3 mmol) was dissolved in dioxane (250 mL). A solution of perchloric acid (5.83 mL of 70% perchloric acid added to 25 mL of H₂O; 18.4 mL of resulting solution used) and water (12.5 mL) were added. The flask was wrapped in foil and cooled in a water-ice bath over 15 min. N-bromoacetamide (12.5 g, 90.6 mmol) was added in portions over 15 minutes. The mixture was removed from the ice bath and stirred for 30 minutes, and then cooled in a water-ice bath before being quenched with 150 mL of 1% sodium thiosulfate solution. The product was extracted with ether 3 times, washed with additional 1% sodium thiosulfate solution until the color had been removed (1-2 washes), 1 wash with water and 1 wash with brine. The organic layer was dried over sodium sulfate, the solvent was removed in vacuo and the material was purified by recrystallization from acetone and water to yield the product as a white solid (15.9 g, 52% yield).

The TLC analysis gave an R_{f}=0.40 in 1:4 EtOAc:Hexane, and the NMR spectrum matched literature reports.

### Synthesis of Compound 7

The synthesis of (3S,5R,6R,8S,9S,10R,13R,14S,17R)-5-bromo-13-methyl-17-((R)-6-methylheptan-2-yl)hexadecahydro-6,10-(epoxymethano)cyclopenta[a]phenanthren-3-yl acetate (3-acetoxy-5-bromo-6-19-oxidocholestane, “compound 7”) proceeded as follows:

Compound 6 (7.7 g, 14.65 mmol) was added to an oven dried flask, and suspended in cyclohexane (150 mL). To this solution, lead tetraacetate (8.12 g, 18.31 mmol), iodine (1.90 g, 7.50 mmol) were added while stirring. The flask was then heated to reflux, and stirred for 2 h. The reaction mixture was cooled to room temperature and quenched 150 mL of 1% sodium thiosulfate solution. The product was extracted with ether 3 times, washed with additional 1% sodium thiosulfate solution until the color had been removed (1-2 washes), 1 wash with water and 1 wash with brine. The organic layer was dried over sodium sulfate, the solvent was removed in vacuo and the material was purified by recrystallization from hexanes, yielding a clear pale yellow residue (5.73 g, 75% yield).

The TLC analysis gave an R_{f}=0.51 in 1:4 EtOAc:Hexane, and the NMR spectrum matched literature reports.

### Synthesis of Compound 8

The synthesis of (3S,8S,9S,10S,13R,14S,17R)-10-(hydroxymethyl)-13-methyl-17-((R)-6-methylheptan-2-yl)-2,3,4,7,8,9,10,11,12,13,14,15,16,17-tetradecahydro-1H-cyclopenta[a]phenanthren-3-yl acetate (3-acetoxy-19-hydroxy-5-cholestene, “compound 8”) proceeded as follows:

Compound 7 (0.2248 g, 0.43 mmol) was dissolved in a solution of acetic acid and water (15:1, 4.32 mL). Activated zinc powder (0.8422 g, 12.881 mmol) was added while stirring. The reaction was then stirred for 21 hours, poured into 35 mL of dichloromethane, and filtered. The filtrate was extracted with an additional 30 mL of dichloromethane. The combined organic layers were washed with brine, and dried over sodium sulfate. The product was purified by flash chromatography (20 g, 1:4 EtOAc:hexane) yielding a solid white residue (0.1057 g, 55.7%).

The TLC analysis gave an R_{f}=0.38 in 1:4 EtOAc:Hexane, and the NMR spectrum matched literature reports.

### Synthesis of Compound 9

The synthesis of (3S,8S,9S,10S,13R,14S,17R)-13-methyl-17-((R)-6-methylheptan-2-yl)-10-((tosyloxy)methyl)-2,3,4,7,8,9,10,11,12,13,14,15,16,17-tetradecahydro-1H-cyclopenta[a]phenanthren-3-yl acetate; (3-acetoxy-19-tosyloxy-5-cholestene, “compound 9) proceeded as follows:

Compound 8 (0.5620 g, 1.264 mmol) was dissolved in dichloromethane (4.14 mL). Dimethylaminopyridine (0.8492 g, 6.95 mmol), and tosyl chloride (1.2049 g, 6.32 mmol) were added. The mixture was stirred for 72 hours, and then partitioned between H₂O and dichloromethane. The dichloromethane layer was separated and washed with saturated aqueous ammonium chloride solution, and brine. The organic layer was dried over sodium sulfate, and purified by flash chromotography on an activated magnesium silicate, Florisil®, column (20 g, 1:9 EtOAc:Hexane) yielding a white solid (0.4025 g, 53% yield).

The NMR spectrum matched literature reports.

### Synthesis of 19-Fluoro-Cholesterol

The synthesis of 19-fluoro-cholesterol proceeded as follows:

Compound 8 (0.0280 g, 0.047 mmol) was dissolved in MeCN (1 mL). TBAF(Pin)₂ (0.0470 g, 0.093 mmol) was added and the reaction was heated at 70° C. for 2 h. The reaction was cooled and ether and water were added to quench the reaction. After extraction the material was deprotected by dissolving the material in a 1:1 mixture of DCM and methanol (1 mL). Potassium carbonate (K₂CO₃) was added and the reaction was stirred overnight. The product was filtered to remove remaining K₂CO₃ and any solids. Deprotection was complete.

An NMR spectrum was obtained to confirm the structure.

### Example 3—Synthesis of Fluorinated Cholesterol

Beginning with a commercially available epoxy-cholesterol (5,6-epoxycholesterol (5α,6α):(5β,6β)), the inventors successfully opened the epoxide ring to fluorinate either the 5 or 6 position.

The scheme of the synthesis of the fluorinated cholesterol is shown below:

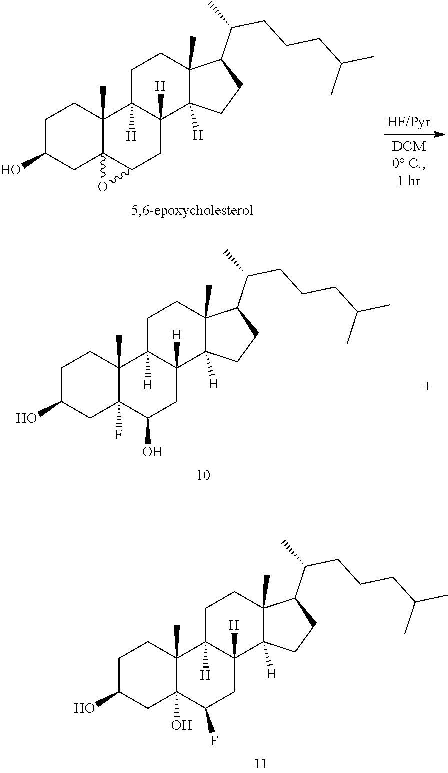

The synthesis of (3S,5R,6R,8S,9S,10R,13R,14S,17R)-5-fluoro-10,13-dimethyl-17-((R)-6-methylheptan-2-yl)hexadecahydro-1H-cyclopenta[a]phenanthrene-3,6-diol (5-fluoro-cholesterol, “compound 10”) and (3S,5R,6R,8S,9S,10R,13R,14S,17R)-6-fluoro-10,13-dimethyl-17-((R)-6-methylheptan-2-yl)hexadecahydro-1H-cyclopenta[a]phenanthrene-3,5-diol (6-fluoro-cholesterol, “compound 11”) proceeded as follows:

A 15 mL falcon tube was charged with 5,6-epoxycholesterol (402 mg, 1.0 mmol; (5α,6α):(5β,6β)=73:27) and DCM (3.0 mL) was added. The resulting solution was cooled in an ice-bath and HF/pyridine 65-70% w/w (280 μL, 10 mmol) was added in one portion after which the cloudy mixture was vigorously stirred at 0° C. for 60 min. The mixture was poured into a mixture of ice and sat. NaHCO₃ solution (25 mL) and extracted with DCM (3×15 mL). The organic layers were washed with brine (25 mL), dried (Na₂SO₄), filtered and concentrated in vacuo. Purification by flash chromatography on a Biotage Isolera Prime system using a KP-SIL-25 g column (eluent DCM/MeOH 97:3) gave compound 10 as a white solid (17 mg, 0.040 mmol, 4%) and compound 11 as a white solid (149 mg, 0.35 mmol, 35%).

The proton NMR spectrum of compound 10 was as follows: ¹H NMR (400 MHz, CDC₃) δ 4.06-3.96 (m, 1H), 3.72 (dt, J=5.3, 2.9 Hz, 1H), 0.90 (d, J=6.4 Hz, 4H), 0.87 (d, J=1.9 Hz, 4H), 0.85 (d, J=1.9 Hz, 4H), 0.68 (s, 3H). The fluorine NMR spectrum of compound 10 was as follows: ¹⁹F NMR (376 MHz, CDC₃) δ −159.81 (d, J=42.6 Hz).

The proton NMR spectrum of compound 11 was as follows: ¹H NMR (400 MHz, CD₃OD) δ 4.18 (dt, J=48.9, 2.7 Hz, 2H), 4.00 (tt, J=11.1, 5.4 Hz, 1H), 3.30 (p, J=1.6 Hz, 1H), 2.04-1.94 (m, 2H), 0.69 (s, 3H). The fluorine NMR spectrum of compound 11 was as follows: ¹⁹F NMR (470 MHz, CDC₃) δ −180.47 (app. dtt, J=48.3, 15.2, 3.5 Hz).

### Example 4—Synthesis of ¹⁸F-Fluorinated Cholesterol

A ¹⁸F-labeled analogue of compound 10 described above was synthesized according to the following reaction scheme:

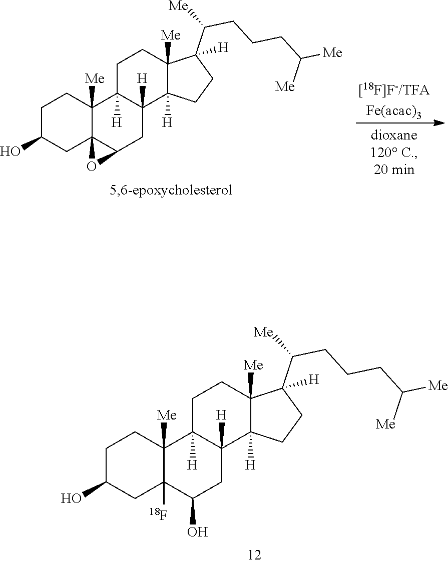

Compound 12 was prepared using a TRACERLab FX_{FN} automated radiochemistry synthesis module (General Electric, GE) in standard configuration using a glassy carbon reactor.

Fluorine-18 was produced by the ¹⁸O(p, n)¹⁸F nuclear reaction using a GE PETTrace cyclotron (a 55 μA beam for 30 minutes generated approx. 1.8 Ci (66.6 GBq) of fluorine-18) and delivered to a GE TRACERLab FX_{FN} automated radiochemistry synthesis module in 2.5 mL bolus of [¹⁸O]H₂O followed by trapping on a Waters QMA SepPak Light Carb cartridge (Waters, order #WAT023525; activated with 10 mL H₂O) as [¹⁸F]F⁻ to remove [¹⁸O]H₂O and other impurities. This was followed by elution (as [¹⁸F]HF) with a solution of TFA in CH₃CN/H₂O 4:1 (0.5 M, 500 μL) from vial 1 into the reactor, which had been charged with Fe(acac)₃ (0.04 mmol, 14 mg). The reactor was then pressurized with argon to approx. 200 kPa (by opening valve 20 for 3 s) and heated at 80° C. for 10 min. The pressure was released by opening valve 24, and the reactor was heated to 110° C. for 10 min under argon flow for azeotropic drying. The drying process was completed by vacuum transfer of CH₃CN (500 μL) from vial 2 to the reactor followed by heating for another 5 min. at 110° C. The reactor was then cooled to 60° C. using compressed air, and a solution of 5,6-epoxycholesterol (0.04 mmol, 18 mg; ratio (5α,6α):(5β,6β)=20:80) in dioxane (500 μL) was added from vial 3 using argon push gas. The reactor was heated to 120° C. and stirred for 20 min under autogenous pressure. After cooling to 50° C. using compressed air, a solution of EtOH:H₂O (4:1, 3.5 mL) was added to the reactor from vial 6 using push gas. The content of the reactor was then pushed with argon through a Waters Al₂O₃ N SepPak Light (activated with 4 mL EtOH) into the intermediate vial and loaded onto a semi-prep HPLC column (Agilent Eclipse XDB 250×9.4 mm 5μ, eluent=80% EtOH/H₂O, flowrate=3 mL/min) for purification. The fraction at Rt=24.1-26.4 min was collected to give 251 mCi (9.29 GBq) of compound 12. An aliquot of the collected fraction was analyzed by radio-HPLC (Phenomenex Luna C18(2) 250×4.6 mm 5μ, eluent=100% CH₃CN) to determine radiochemical identity and purity.

### Example 5—Synthesis of FNP-59 Precursor

Synthesis of a FNP-59 precursor followed the scheme, below:

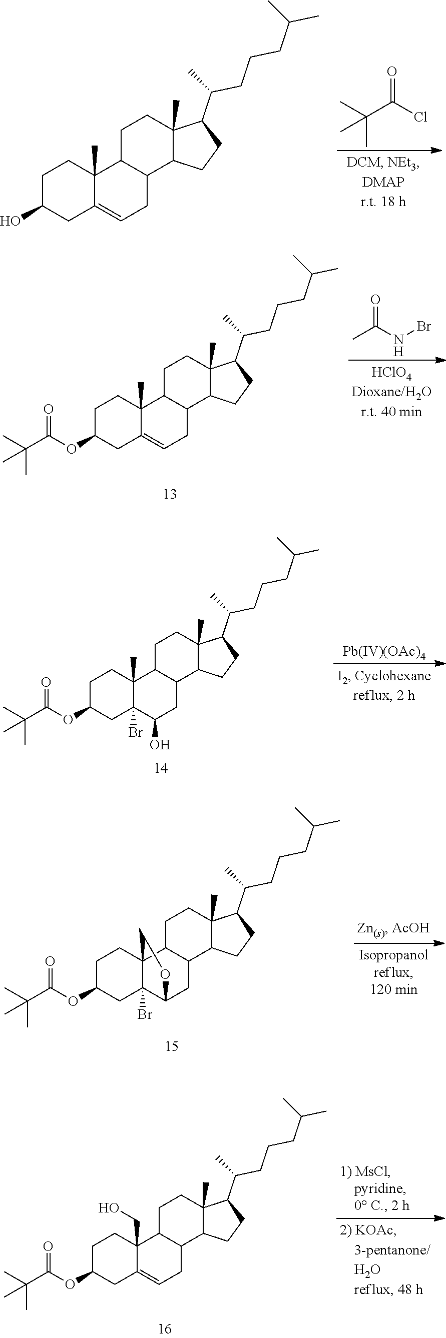

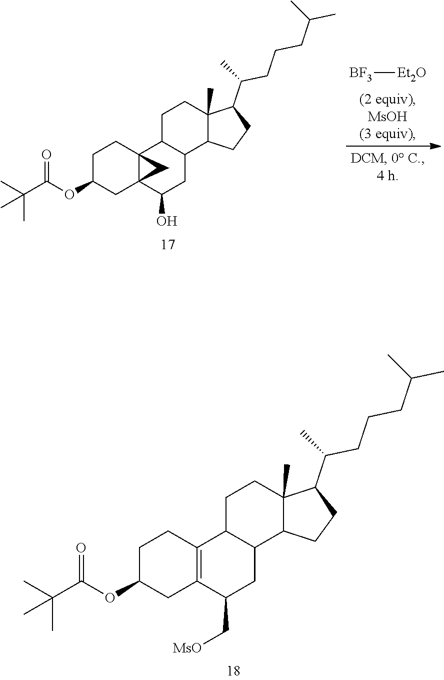

### Synthesis of Compound 13

Cholesterol (10 g, 25.86 mmol) was added to a flame dried flask and dissolved in dichloromethane (50 mL). To this solution, triethylamine (4.32 mL, 31.03 mmol) and dimethylaminopyridine (0.3164 g, 2.59 mmol) were added. The solution was then cooled to 0° C., and pivaloyl chloride (3.5 mL, 28.45 mmol) was added dropwise while stirring. The reaction was then stirred at room temperature for 48 hours. The solvent was removed in vacuo, and the residue was triturated in 75 mL of hot acetone for 10 minutes, and then 5 mL of water was added. The suspension was allowed to cool for 2 hours, and then the liquid was removed by vacuum filtration to give compound 13. TLC R_{F}=0.86, 1:9 EtOAc:Hexane. ¹H-NMR (400.53 MHz, CDCl₃): δ 5.36 (1H, d, J=4.62 Hz, 6-H), 4.56 (1H, m, 3α-H), 1.18 (9H, s, 3β-OPiv). ¹³C-NMR (100.13 MHz, CDCl₃): δ 177.98, 139.77, 122.46, 73.52, 56.67, 56.11, 49.99, 42.30, 39.72, 39.50, 38.59, 38.00, 36.97, 36.60, 36.17, 35.79, 31.88, 28.22, 28.00, 27.65, 27.15, 24.28, 23.82, 22.81, 22.56, 21.03, 19.36, 18.71, 11.84. HR-MS (ESI+) [M+NH₄]⁺ Calculated for C₃₂H₅₄O₂: 488; Found: 488.

### Synthesis of Compound 14

Compound 13 (5 g, 10.62 mmol) was dissolved in dioxane (50 mL). A solution of perchloric acid (6.37 mL of 0.5M) was added while stirring. The reaction vessel was then wrapped in foil, and N-bromoacetamide was added slowly over 5 minutes. The reaction was stirred for 40 minutes before being quenched by the addition of 10% sodium thiosulfate solution (50 mL). The mixture was then extracted with diethyl ether 3 times, and the resulting organic layer was isolated and dried over sodium sulfate. The solvent was removed in vacuo, and the material was purified by flash chromatography (20 g silica, 1:19 EtOAc:Hexane) yielding Compound 14. TLC R_{F}=0.42, 1:9 EtOAc:Hexane. ¹H-NMR (400.53 MHz, CDCl₃): δ 5.44 (1H, m, 3α-H), 4.19 (1H, s, 6β-OH), 2.47 (1H, m, 6α-H), 1.18 (9H, s, 3β-OPiv). ¹³C-NMR (100.13 MHz, CDCl₃): δ 177.98, 86.82, 75.79, 71.78, 56.07, 55.70, 47.42, 42.68, 40.36, 39.65, 39.49, 38.61, 38.31, 36.11, 35.75, 35.12, 34.59, 30.57, 28.19, 28.00, 27.16, 26.23, 24.05, 23.79, 22.81, 22.55, 21.31, 18.67, 18.08, 12.19. HR-MS (ESI+) [M+NH₄]⁺ Calculated for C₃₂H₅₅BrO₃: 584; Found: 584.

### Synthesis of Compound 15

Compound 14 (2.88562 g, 5.031 mmol) was added to a flame dried flask and dissolved in cyclohexane (50 mL). To this solution, lead tetraacetate (2.7884 g, 6.289 mmol) and iodine (0.6386 g, 2.516 mmol) were added while stirring. The reaction was then stirred at 90° C. for 2 hours. It was then allowed to cool to room temperature, and then filtered. The filter was then washed with diethyl ether. The filtrate was then partitioned with a 10% solution of sodium thiosulfate, and the mixture was extracted with additional diethyl ether. The organic layer was then washed with water and brine. The solvent was removed in vacuo to give compound 15, which was used directly in the next reaction. TLC R_{F}=0.69, 1:9 EtOAc:Hexane.

### Synthesis of Compound 16

Compound 15 (2.5381 g, 4.48 mmol) was dissolved in isopropanol (45 mL) and glacial acetic acid (2.6 mL). Zinc powder was activated by being stirred under vacuum at 80° C. The activated zinc (1.6125 g, 24.66 mmol) was then added while stirring. The reaction was then stirred at 90° C. for 30 minutes, before being removed from heat, and allowed to stir at room temperature for an additional 18 hours. The resulting mixture was allowed to settle, and the liquid was decanted off. The solid was then decanted 3 more times with dichloromethane. The solvent was removed in vacuo and the material was purified by flash chromatography (20 g silica, 1:19 EtOAc:Hexane) to give compound 16. TLC R_{F}=0.28, 1:9 EtOAc:Hexane. ¹H-NMR (400.53 MHz, CDCl₃): δ 5.76 (1H, d, J=4.15 Hz, 6-H), 4.61 (1H, m, 3α-H), 3.85 (1H, d, J=11.28 Hz, 19-H), 3.63 (1H, t, J=9.17 Hz, 19-H), 1.17 (9H, s, 3β-OPiv). ¹³C-NMR (100.13 MHz, CDCl₃): δ 177.94, 134.66, 128.10, 72.96, 62.68, 57.53, 56.08, 50.25, 42.50, 41.60, 39.99, 39.49, 38.59, 38.08, 36.15, 35.77, 33.34, 33.02, 31.26, 28.23, 27.99, 27.12, 24.08, 23.82, 22.82, 22.56, 21.77, 18.69, 12.19. HR-MS (ESI+) [M+H]⁺ Calculated for C₃₂H₅₄O₃: 487; Found: 487. [M+NH₄]⁺ Calculated for C₃₂H₅₄O₃: 504; Found: 504 [M+Na]⁺ Calculated for C₃₂H₅₄O₃: 509; Found: 509.

### Synthesis of Compound 17

Compound 16 (1.1128 g, 2.286 mmol) was dissolved in pyridine (11.43 mL). The reaction was cooled to 0° C. and methanesulfonyl chloride (0.885 mL, 11.43 mmol) was added dropwise, and the reaction was stirred at 0° C. for 2 hours. The reaction was then quenched with 20 mL of cold water, and extracted with dichloromethane 3 times. The organic layer was then washed with brine, and the solvent was removed in vacuo. The resulting residue was resuspended in 3-pentanone (76 mL), and a solution of potassium acetate (1.2339 g in 23 mL water) was added. The reaction was then stirred at 120° C. for 48 hours. When TLC indicated the consumption of starting material, the reaction was allowed to cool to room temperature, and extracted with ethyl acetate. The material was loaded onto Florosil gel, and purified by flash chromatography (20 g silica, 1:19 EtOAc:Hexane) to give compound 17. TLC R_{F}=0.34, 1:4 EtOAc:Hexane. ¹H-NMR (400.53 MHz, CDCl₃): δ 4.74-4.66 (1H, m, 3α-H), 4.10 (1H, br), 2.16-2.11 (1H, m), 2.06-1.98 (2H), 1.91-1.68 (5H), 1.57-1.43 (4H), 1.37-1.25 (3H), 1.22-1.18 (3H), 1.16 (9H, s, 3β-OPiv), 1.13-0.99 (9H), 0.91-0.85 (10H), 0.65 (3H, s), 0.31 (1H, d, J=4.9 Hz). ¹³C-NMR (100.13 MHz, CDCl₃): δ 178.06, 73.92, 70.05, 56.38, 54.64, 48.19, 43.03, 39.96, 39.86, 39.48, 38.62, 37.24, 36.12, 35.72, 29.38, 28.18, 28.00, 27.46, 27.13, 26.66, 26.10, 25.11, 23.91, 23.81, 22.81, 22.55, 18.65, 15.59, 12.25. HR-MS (ESI+) [M+Na]⁺ Calculated for C₃₂H₅₄O₃: 509; Found: 509. [2M+Na]⁺ Calculated for C₆₄H₁₀₈O₆: 996; Found 996.

### Synthesis of Compound 18 (FNP-59 Precursor)

Compound 17 (0.4000 g, 0.82 mmol) was dissolved in dichloromethane (8 mL) under argon. Methanesulfonic acid (0.16 mL, 2.46 mmol) was added while stirring. The reaction mixture was cooled to 0° C., and boron trifluoride diethyl etherate (0.20 mL, 1.64 mmol) was added, and the reaction was stirred for 4 hours. The reaction was then extracted with dichloromethane, and washed with saturated sodium bicarbonate solution and brine.

The combined aqueous layer was then extracted with diethyl ether 3 times. The combined organic layers were then dried over sodium sulfate, the material was loaded onto Florosil gel, and purified by flash chromatography (20 g Florosil, 1:9 EtOAc:Hexane) to give compound 18. TLC R_{F}=0.29, 1:4 EtOAc:Hexane). ¹H-NMR (400.53 MHz, CDCl₃): δ 4.94 (1H, m, 3α-H), 4.18 (1H, m, 6β-CH₂), 4.07 (1H, t, J=9.79 Hz, 6β-CH₂), 2.98 (3H, t, J=6.71 Hz, 6β-OMs). ¹³C-NMR (100.13 MHz, CDCl₃): δ 178.09, 135.67, 121.61, 70.66, 68.97, 56.29, 54.74, 46.48, 43.08, 40.12, 39.87, 39.47, 38.74, 37.43, 36.11, 35.74, 34.64, 33.68, 28.55, 28.27, 27.98, 27.14, 25.64, 24.42, 23.78, 23.60, 22.81, 22.55, 18.62, 12.27. HR-MS [M+NH₄]⁺ Calculated for C₃₃H₅₆O₅S: 582; Found 582.

### Fluorination of FNP-59 Precursor

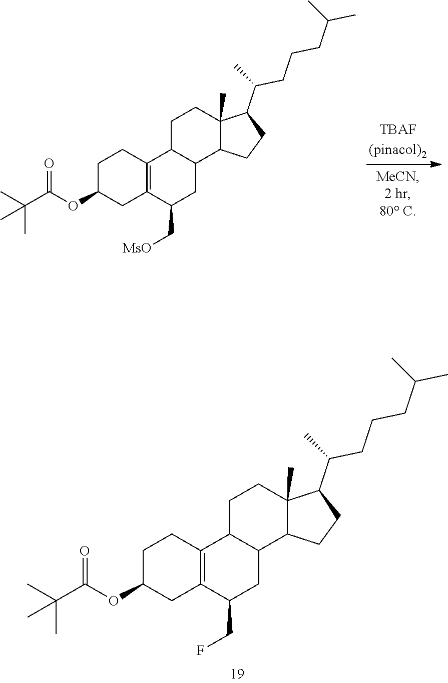

Compound 18 (0.1050 g, 0.186 mmol) was dissolved in acetonitrile (1 mL). Tetrabutylammonium fluoride bis(pinacol) (0.18523 g, 0.372 mmol) was added while stirring. The reaction was then heated to 80° C. and stirred for 2 hours. It was then allowed to cool to room temperature, and extracted with diethyl ether. The material was then loaded onto Florosil gel, and purified by flash chromatography (20 g Florosil, 1:19 EtOAc:Hexane) to give compound 19. TLC R_{F}=0.92, 1:4 EtOAc:Hexane. ¹H-NMR (400.53 MHz, CDCl₃): δ 5.08 (1H, m, 3α-H), 4.70 (2H, d, J=48.99 Hz, 6β-CH₂), 3.58 (1H, m, 6α-H), 1.17 (9H, s, 3β-OPiv). ¹⁹F-NMR (376.87 MHz, CDCl₃): δ −227.84 (m).

### Example 6—Radiosynthesis of [¹⁸F]NP-59

The synthesis followed the scheme, below:

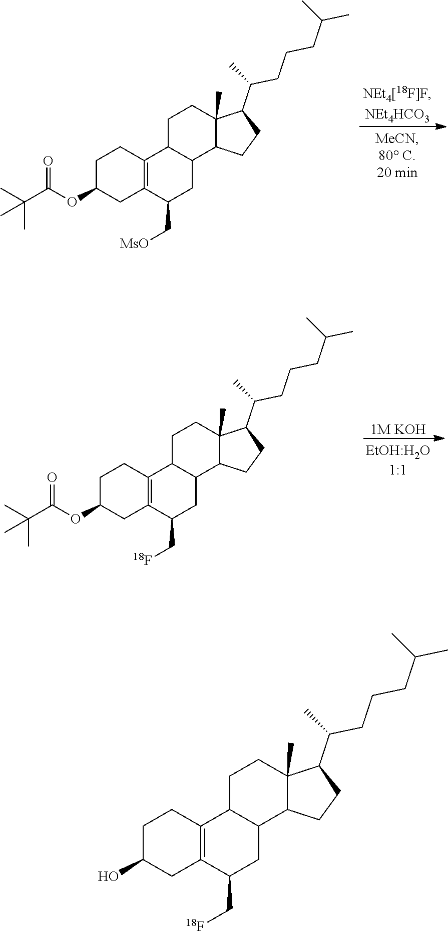

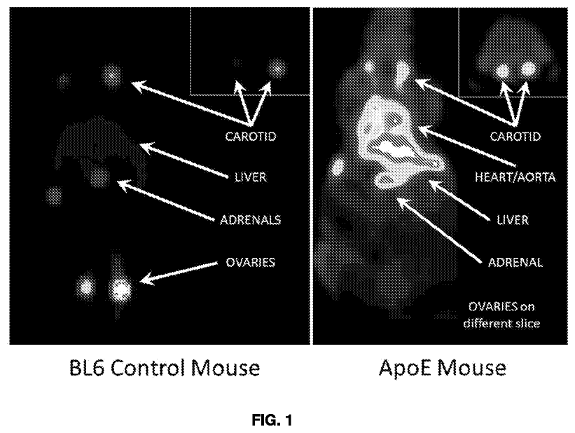

The synthesis of [¹⁸F]NP-59 was accomplished using a General Electric (GE) TRACERLab FX_{FN} synthesis module loaded as follows: Vial 1: 500 μL of 23 mg/mL tetraethylammonium bicarbonate in water; Vial 2: 1000 μL of acetonitrile (or other solvent with H₂O azeotrope, e.g. ethanol); Vial 3: 5 mg precursor in 1000 μL acetonitrile (or other polar aprotic solvent, e.g. DMSO); Vial 4: 1000 μL of a 1M potassium hydroxide solution in H₂O:Ethanol (1:1). [¹⁸F]Fluoride was produced via the ¹⁸O(p,n)¹⁸F nuclear reaction with a GE PETtrace cyclotron equipped with a high-yield fluorine-18 target. [¹⁸F]Fluoride was delivered in a bolus of [¹⁸O]H₂O to the synthesis module and trapped on a QMA-Light sep-pak cartridge to remove [¹⁸O]H₂O. [¹⁸F]Fluoride was then eluted into the reaction vessel with tetraethylammonium bicarbonate (11.5 mg in 500 μL of water). Acetonitrile (1 mL) was added to the reaction vessel, and the [¹⁸F]fluoride was azeotropically dried by heating the reaction vessel to 100° C. and drawing full vacuum. After this time, the reaction vessel was subjected to both an argon stream and a simultaneous vacuum draw at 100° C. The solution of FNP-59 precursor (compound 18) in acetonitrile (or other polar aprotic solvent, e.g., DMSO) (5 mg in 1000 μL) was added to the dried [¹⁸F]fluoride, and was heated at 90° C. with stirring for 20 min. Subsequently, the reaction mixture was cooled to 50° C., and the 1M potassium hydroxide solution was added. The reaction mixture was heated at 110° C. for 25 minutes. The reaction mixture was then cooled to 50° C. and removed from the synthesis module for analysis. HPLC was performed using an Phenomenex Ultracarb ODS (30) 250×4.6 mm, 5μ column with a mobile phase of 90% EtOH at 1 mL/min. UV peaks were detected at 212 nm.

[¹⁸F]NP-59 was injected into a control BL6 mouse and an ApoE mouse of similar weight. Equivalent activities were injected into each mouse, and PET images taken approximately 60 minutes after injection are shown in FIG. 1. The images are PET maximal intensity projection images in the oblique coronal plane to obtain relevant anatomic structures. In the upper right-hand corner of each image are axial images through the carotid artery.

FIG. 1 shows differential uptake between the mice, with higher uptake in the ApoE mouse, known to have atherosclerotic disease. The images show uptake of the compound in the liver, adrenal glands, and liver. The ApoE mouse has higher background uptake even though the mice are of similar weight, and identical amounts of tracer were injected. Therefore, Example 6 demonstrates that [¹⁸F]NP-59 can be used to image and identify altered cholesterol metabolism and atherosclerotic disease.

### REFERENCES

1) Paillasse M. R.; Saffon, N.; Gornitzka, H; Silvente-Poirot, S.; Poirot, M; de Medina, P. J. Lipids. Res. 2012, 53, 718-725

What is claimed:

1. A compound having the structure of Formula (I): 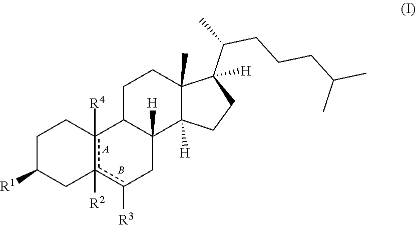
wherein:
R¹ is OH or OP;
R², when present, is OH or X;
R³ is H, OH, X, CH₂—X, or CH₂-LG;
R⁴, when present, is C₁₋₆ alkyl, C₁₋₆ alkylene-X, or C₁₋₆ alkylene-LG;
X is a halogen;
P is an alcohol protecting group; and
LG is a leaving group;
each of bond A and bond B is a single or a double bond and only one of bond A and bond B can be a double bond;
with the proviso that:
at least one X or LG is present; and if LG is present, R¹ is OP;
if one of R² and R³ is F and the other OH, then the F is ¹⁸F; and
the compound is not:

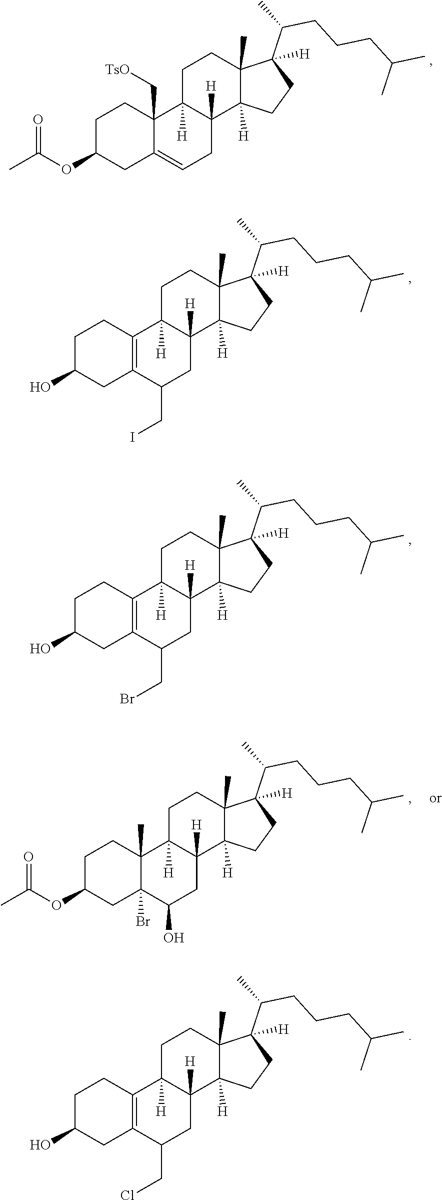

2. The compound of claim 1, wherein X is F or I.

3. The compound of claim 2, wherein X is ¹⁸F.

4. The compound of claim 2, wherein X is ¹²⁴I or ¹³¹I.

5. The compound of any one of claims 1 to 4, wherein R¹ is OH.

6. The compound of any one of claims 1 to 4, wherein R¹ is OP.

7. The compound of claim 6, wherein P is pivaloyl, acetoxy, THP, or MOM.

8. The compound of claim 7, wherein P is THP or MOM.

9. The compound of any one of claims 1 to 8, wherein R² is X.

10. The compound of any one of claims 1 to 8, wherein R³ is X or CH₂—X.

11. The compound of any one of claims 1 to 8, wherein R⁴ is C₁₋₆alkylene-X.

12. The compound of any one of claims 1 to 9 and 11, wherein R³ is CH₂-LG.

13. The compound of any one of claims 1 to 10, wherein R⁴ is C₁₋₆alkylene-LG.

14. The compound of any one of claims 1 to 13, wherein LG is tosyl, a halogen, mesyl, or triflate.

15. The compound of claim 14, wherein LG is tosyl or mesyl.

16. The compound of any one of claims 1 to 15, wherein A is a double bond.

17. The compound of any one of claims 1 to 15, wherein B is a double bond.

18. The compound of any one of claims 1 to 15, wherein each of A and B is a single bond.

19. The compound of any one of claims 1 to 16, having a structure (IA) 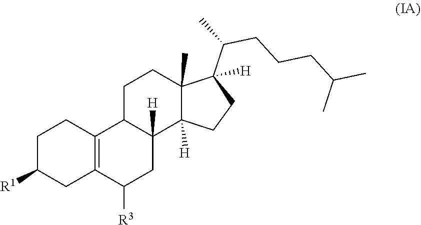
wherein R³ is C₁₋₆ alkylene-X or C₁₋₆ alkylene-LG.

20. The compound of claim 19, wherein R¹ is OP and R³ is CH₂-LG.

21. The compound of claim 19 or 20, wherein P is acetoxy and LG is OTs.

22. The compound of claim 19 or 20, wherein P is pivaloyl, MOM, or THP and LG is OTs or OMs.

23. The compound of claim 22, wherein P is pivaloyl and LG is OMs.

24. The compound of any one of claims 19 to 23, wherein R³ is CH₂—OTs or CH₂—OMs.

25. The compound of claim 19, wherein R¹ is OP and R³ is C₁₋₆ alkylene-X.

26. The compound of claim 19, wherein R¹ is OH and R³ is C₁₋₆ alkylene-X.

27. The compound of claim 25 or 26, wherein R³ is CH₂—X.

28. The compound of any one of claims 1 to 15 and 17, having a structure of formula (IB) 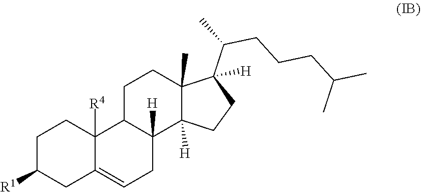
wherein R⁴ is C₁₋₆ alkylene-X or C₁₋₆ alkylene-LG.

29. The compound of claim 28, wherein R¹ is OP and R⁴ is C₁₋₆ alkylene-LG.

30. The compound of claim 28 or 29, wherein P is acetoxy and LG is OTs.

31. The compound of claim 28 or 29, wherein P is MOM or THP and LG is OTs or OMs.

32. The compound of any one of claims 28 to 31, wherein R⁴ is CH₂—OTs or CH₂—OMs.

33. The compound of claim 28, wherein R¹ is OH and R⁴ is C₁₋₆ alkylene-X.

34. The compound of claim 33, wherein R⁴ is CH₂—X.

35. The compound of any one of claims 1 to 13, having a structure of Formula (IC) 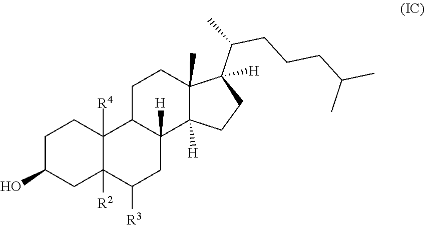
wherein one of R² and R³ is OH and the other is X, and R⁴ is C₁₋₆ alkylene.

36. The compound of claim 35, wherein R⁴ is methyl.

37. The compound of claim 35 or 36, wherein R² is X and R³ is OH.

38. The compound of claim 35 or 36, wherein R² is OH and R³ is X.

39. The compound of claim 1 having a structure selected from the group consisting of:

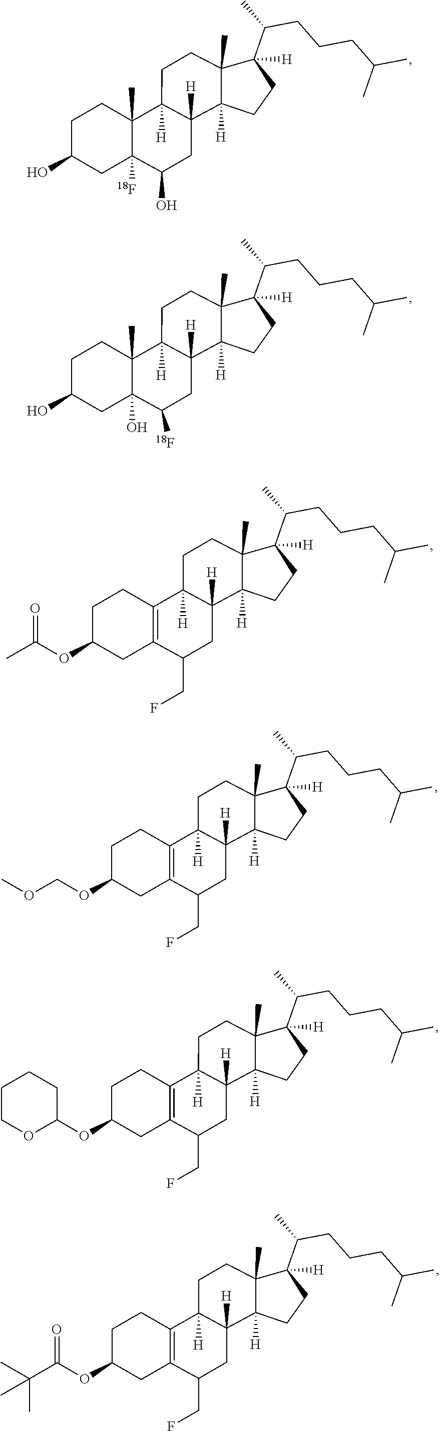

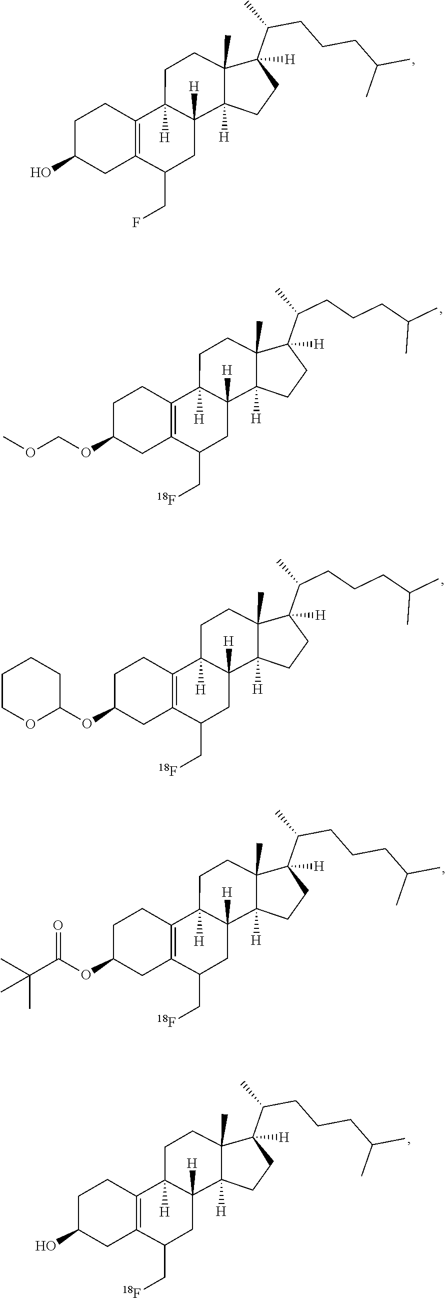

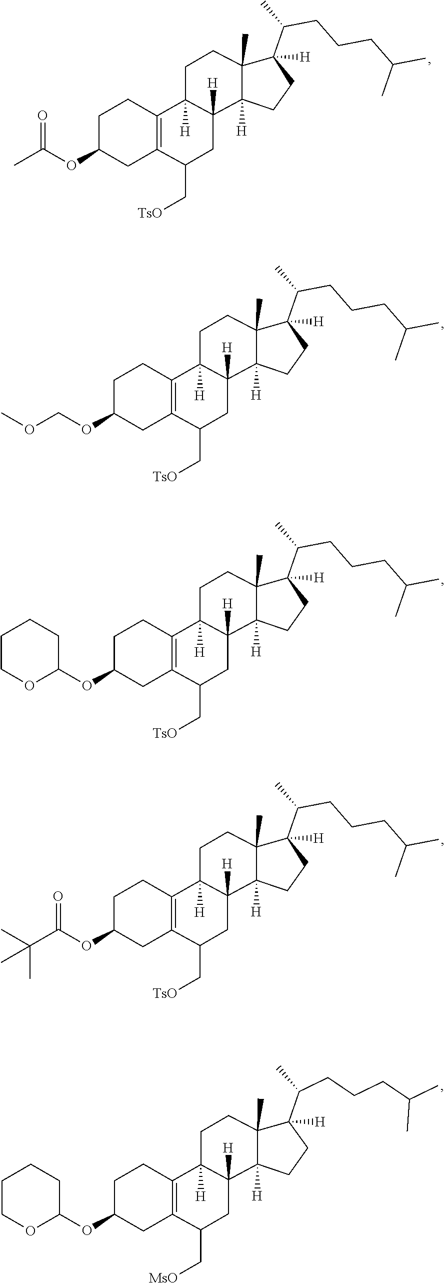

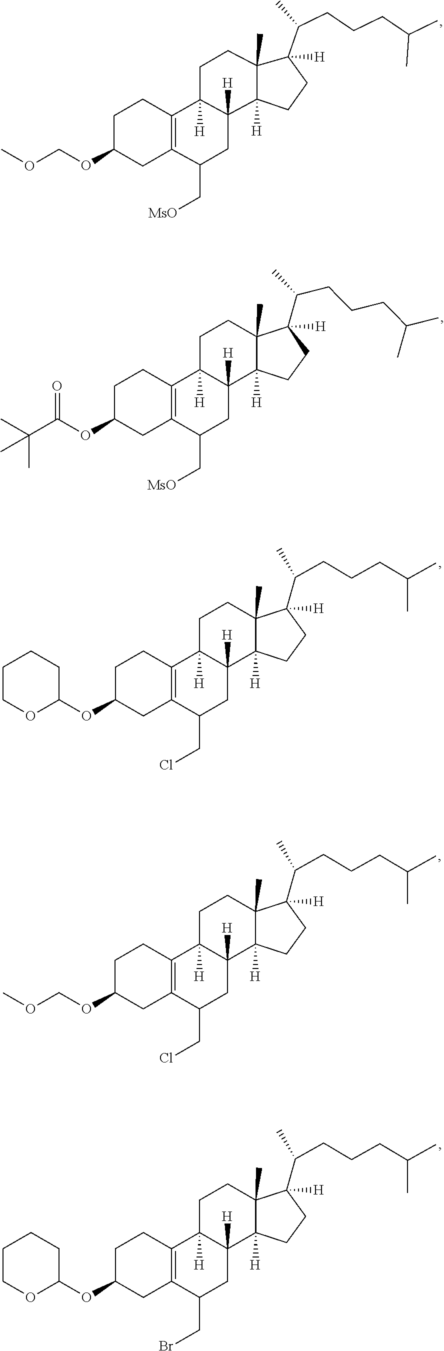

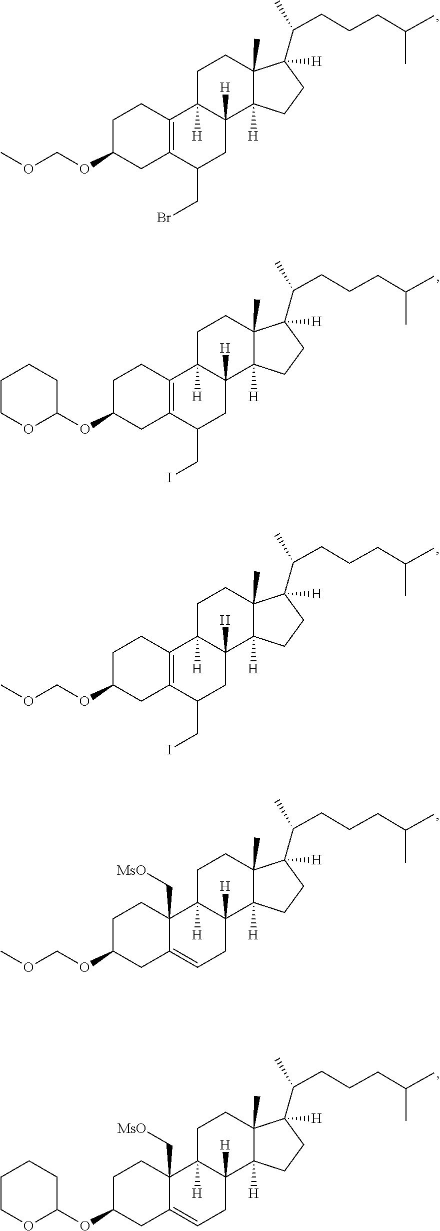

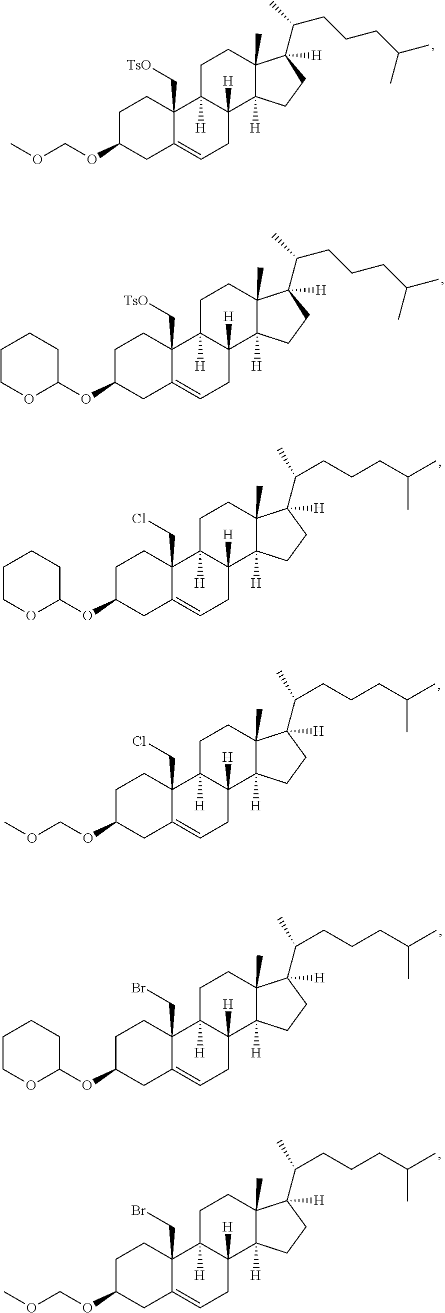

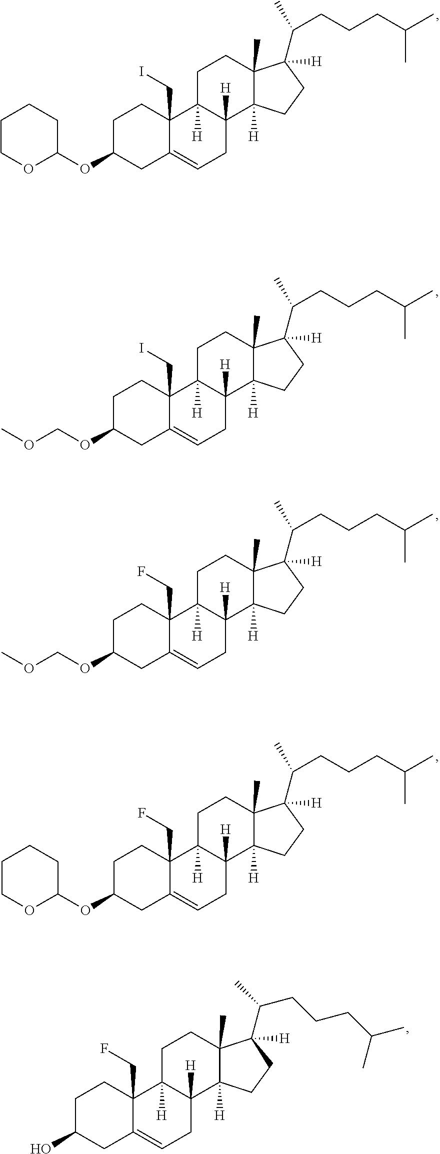

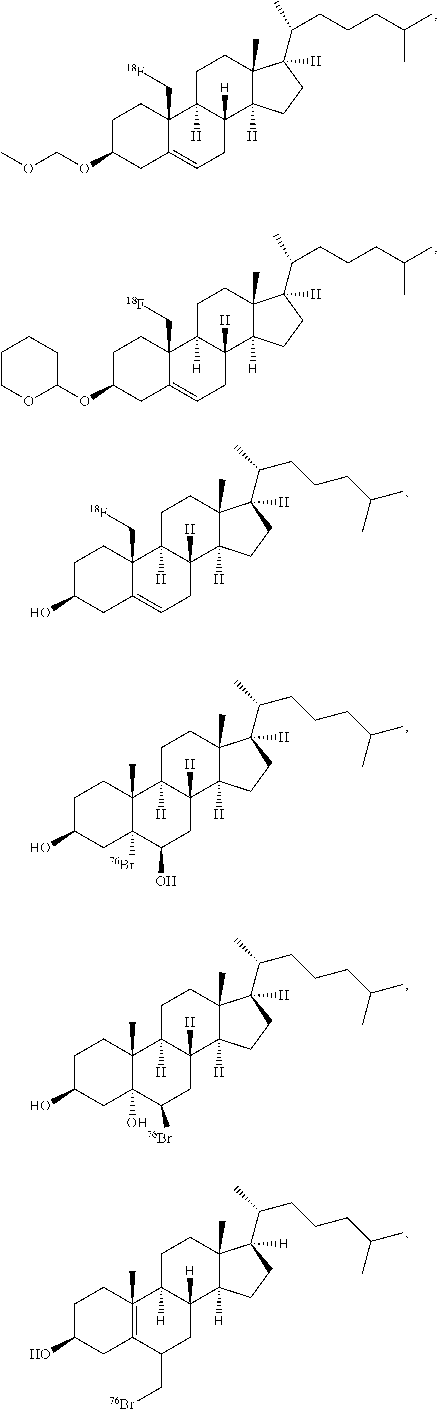

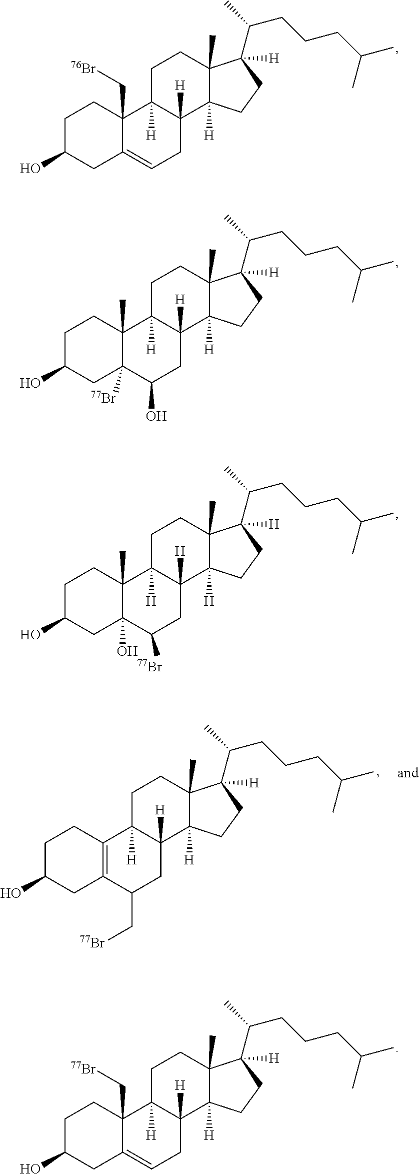

40. The compound of claim 39, having a structure selected from the group consisting of:

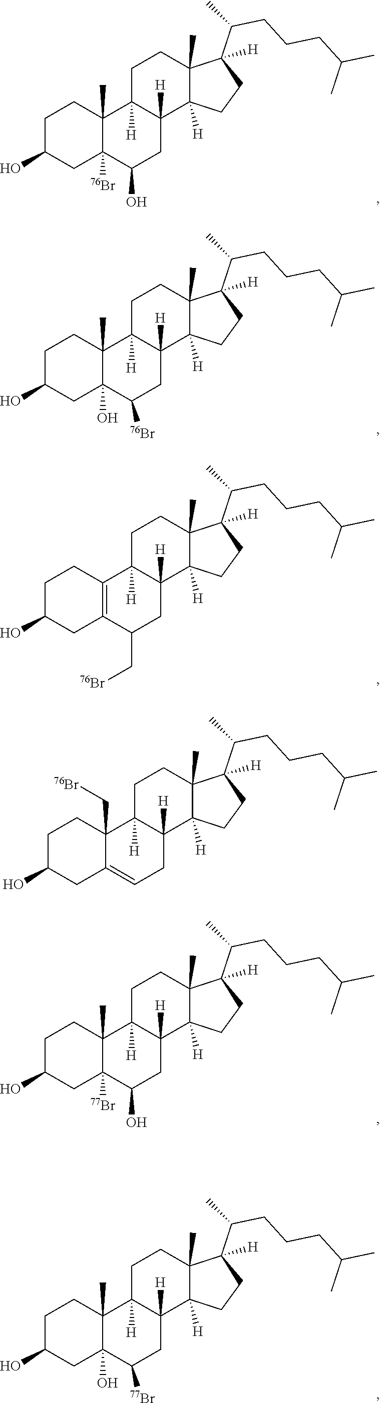

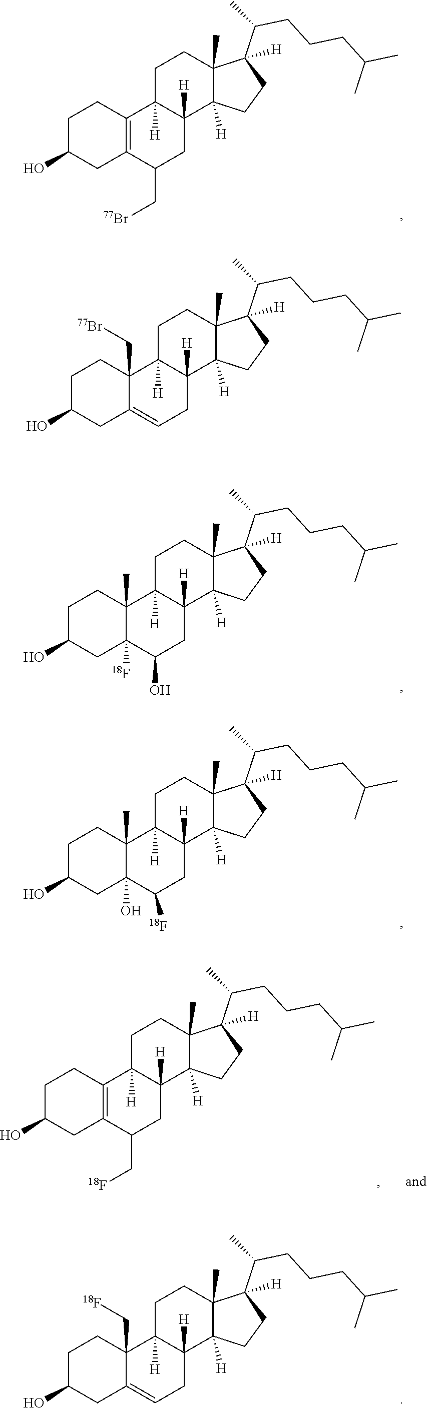

41. The compound of claim 39, having a structure selected from the group consisting of:

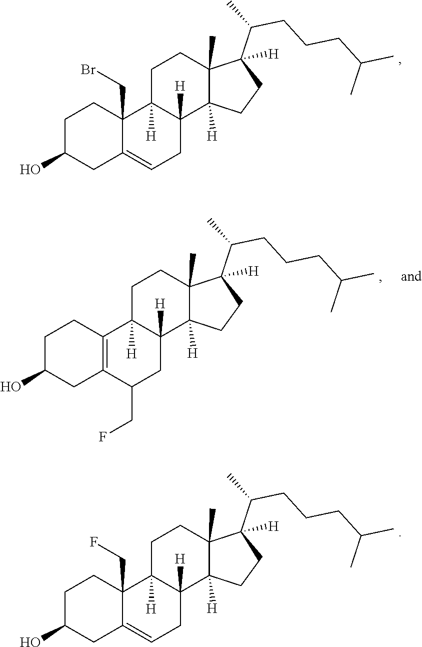

42. A method of preparing a compound having the structure of Formula (II) 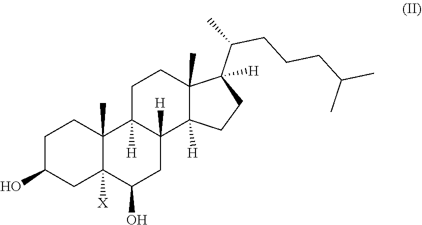
wherein X is ¹⁸F, ⁷⁶Br, or ⁷⁷Br,
comprising:
admixing 5,6-epoxycholesterol and a radiolabeled source under conditions sufficient to form the compound of Formula (II).

43. The method of claim 42, wherein the radiolabeled source comprises fluorine-18, bromine-76, or bromine-77.

44. The method of claim 42 or 43, wherein the admixing step occurs at a temperature of about 50° C. to about 150° C.

45. The method of any one of claims 42 to 44, wherein the admixing step occurs for about 5 minutes to about 30 minutes.

46. A method comprising
administering to a subject the compound of claim 40 or 41; and
subjecting the subject to an imaging modality.

47. The method of claim 46, wherein the imaging modality is selected from the group consisting of positron emission tomography (PET), positron emission tomography/computed tomography (PET/CT), positron emission tomography/magnetic resonance imaging (PET/MRI), single-photon emission computerized tomography (SPECT), and single-photon emission computerized tomography/computed tomography (SPECT/CT).

48. The method of claim 46 or 47, wherein the subject suffers from or is suspected of suffering from Cushing's syndrome, primary aldosteronism, hyperandrogenism, adenoma, pheochromocytoma, an atherosclerotic disease, a disorder of cholesterol metabolism and distribution, or ectopic cholesterol production.

49. The method of claim 48, wherein the adenoma is an adrenal adenoma.

50. The method of claim 48, wherein the atherosclerotic disease comprises vulnerable plaque.

51. The method of claim 50, wherein the patient has vulnerable plaque and the imaging step identifies the vulnerable plaque.

52. The method of any one of claims 46 to 51, wherein the subject is subjected to the imaging modality at a point in time ranging from about 0.5 hours to about 7 days after administration of the compound.

53. The method of any one of claims 46 to 52, wherein the subject is subjected to the imaging modality at a point in time ranging from about 0.1 hours to about 12 hours after administration of the compound.

54. The method of any one of claims 46 to 53, further comprising administering to the subject a drug or a steroid prior to the administration of the compound.

55. The method of any one of claims 46 to 54, wherein the subject suffers from or is suspected of suffering from an Akt-associated disorder.

56. A method comprising admixing a cholesterol epoxide with a metal catalyst and a fluorine-18 source to form a α,β-hydroxy fluoride cholesterol compound, wherein the fluorine-18 source comprises H-¹⁸F.

57. The method of claim 56, wherein the fluorine-18 source is present in a molar equivalent of about 1 to about 2 relative to the epoxide.

58. The method of claim 56 or 57, wherein the metal catalyst comprises a metal selected from the group consisting of iron and gallium.

59. The method of any one of claims 56 to 58, wherein the metal catalyst comprises ferric acetylacetonate.

60. The method of any one of claims 56 to 59, wherein the admixing step occurs for less than 1 hour.

61. The method of any one of claims 56 to 60, wherein the admixing step occurs for about 5 to about 45 minutes.

62. The method of any one of claims 56 to 61, wherein the admixing step occurs at a temperature of about 50° C. to about 150° C.

63. A method comprising
admixing cholesterol and an acyl chloride to form cholest-5-en-3-acylate;
reacting cholest-5-en-3-acylate with N-bromoacetamide to form a 5-bromocholestan-6-hydroxy-3-acylate;
reacting the 5-bromocholestan-6-hydroxy-3-acylate with lead tetraacetate to form a 5-bromocholestan-6(19)-oxo-3-acylate;
reacting 5-bromocholestan-6(19)-oxo-3-acylate with activated zinc to form a cholest-5-en-19-hydroxy-3-acylate;
reacting the cholest-5-en-19-hydroxy-3-acylate with mesyl chloride then potassium acetate to form (3S,5R,10S,13R,17R)-6-hydroxy-13-methyl-17-((R)-6-methylheptan-2-yl)tetradecahydro-6H-5,10-methanocyclopenta[a]phenanthren-3-yl acylate; and
reacting (3S,5R,10S,13R,17R)-6-hydroxy-13-methyl-17-((R)-6-methylheptan-2-yl)tetradecahydro-6H-5,10-methanocyclopenta[a]phenanthren-3-yl acylate with boron trifluoride and methanesulfonic acid to form 6-methyl(methanesulfonyl)-19-norcholest-5(10)-en-3-yl acylate.

64. The method of claim 63, further comprising
reacting 6-methyl(methanesulfonyl)-19-norcholest-5(10)-en-3-yl acylate with an 18-F source then treating with a strong base (optionally potassium hydroxide) to form ¹⁸-FNP-59.

65. The method of claim 63, further comprising
reacting 6-methyl(methanesulfonyl)-19-norcholest-5(10)-en-3-yl acylate with TBAF to form FNP-59.

66. The method of any one of claims 63-65, wherein the acyl chloride comprises pivaloyl chloride, benzoyl chloride, or acetyl chloride.

67. The method of claim 66, wherein the acyl chloride comprises pivaloyl chloride.
